(19) United States
(12) Patent Application Publication
(10) Pub. No.: US 2012/0095063 A1
(43) Pub. Date: Apr. 19, 2012

(54) METHODS AND COMPOSITIONS FOR TREATMENT OF ISCHEMIC CONDITIONS AND CONDITIONS RELATED TO MITOCHONDRIAL FUNCTION

(76) Inventors: Francisco Villareal, Chula Vista, CA (US); Pam R. Taub, Rancho Santa Fe, CA (US); Alan S. Maisel, Solana Beach, CA (US); George F. Schreiner, Los Altos Hills, CA (US); Anne Murphy, Encinitas, CA (US); Katrina Yamazaki, Diamond Bar, CA (US); Guillermo Ceballos, Lindavista (MX)

(21) Appl. No.: 13/264,935

(22) PCT Filed: Apr. 17, 2010
(86) PCT No.: PCT/US10/31530

§ 371 Date: Oct. 31, 2011

Related U.S. Application Data
(60) Provisional application No. 61/170,557, filed on Apr. 17, 2009.
(60) Provisional application No. 61/243,501, filed on Sep. 17, 2009.

(51) Int. Cl. A61K 31/353; A61P 3/00; A61P 9/12; A61P 13/12; A61P 7/00; A61P 9/04; A61P 3/04; A61P 5/48; A61P 21/00; A61K 31/4406; A61P 25/28

(52) U.S. Cl. 514/355; 514/456

(57) ABSTRACT

The present invention relates to compositions and methods for prophylactic and/or therapeutic treatment of conditions related to mitochondrial function. In various aspects, the present invention comprises administering one or more compounds selected from the group consisting of epicatechin, an epicatechin derivative, catechin, a catechin derivative, nicorandil, and a nicorandil derivative in an amount effective to stimulate mitochondrial function in cells. The methods and compositions described herein provide for reducing infarct size in the heart following permanent ischemia or ischemia/reperfusion (IR) event or method for delaying, attenuating or preventing adverse cardiac remodeling, and can assist in prevention of impaired mitochondria biogenesis and thus prevention of the consequences of impaired mitochondrial biogenesis in various diseases and conditions, as well as provide for the active therapy of mitochondrial depletion that may have already occurred.

The present application claims priority from U.S. Provisional Patent Application 61/170,557 filed Apr. 17, 2009, and U.S. Provisional Patent Application 61/243,501 filed Sep. 17, 2009, each of which is hereby incorporated in its entirety including all tables, figures, and claims.

### BACKGROUND

The following discussion of the background of the invention is merely provided to aid the reader in understanding the invention and is not admitted to describe or constitute prior art to the present invention.

The present patent application relates to treatment and prevention of acute injuries, and prevention or reversal of states of chronic mitochondrial depletion or dysfunction.

Ischemic organ injury, and the related condition of ischemia/reperfusion injury, is accompanied by changes in signaling molecules and metabolic effectors that can, independently or in concert, trigger cell death in its various forms. These include changes in intracellular pH, calcium, ceramide, free radicals, hypoxia and adenosine triphosphate (ATP) depletion. While all of these factors may be significantly altered as a consequence of acute necrotic cell death, they can also be specific effectors of apoptotic death under certain circumstances.

The contributions of apoptotic cell death and cellular necrosis to functional deterioration of the organ in ischemic conditions such as myocardial infarction and stroke are well established. Myocardial infarctions generally result in an immediate depression in ventricular function due to myocardial cell necrosis and apoptosis. These infarctions are also likely to expand, provoking a cascading sequence of myocellular and structural events which ultimately result in adverse cardiac remodeling. In many cases, this progressive myocardial infarct expansion and adverse ventricular remodeling (thinning of left ventricular wall, scar tissue formation) leads to deterioration in ventricular function and heart failure.

Ischemic renal injury has been traditionally associated with tubular cell necrosis along with obstructive cast formation, disruption of architecture, and a significant inflammatory response. More recently did apoptosis emerge as a significant mode of cell death during ischemic renal injury. While the contribution of apoptotic cell death to functional deterioration of the organ is obvious in conditions like myocardial infarction and stroke, it is less clear how apoptotic dropout of tubular cells can impact glomerular filtration rate (GFR). Nevertheless, recent reports have demonstrated that interference with the apoptotic program does translate into a protective effect on renal function.

Despite considerable advances in the diagnosis and treatment of conditions related to apoptosis and cellular necrosis, there remains a need in the art for prophylactic and therapeutic approaches for the treatment of these conditions.

The phrase “conditions related to mitochondrial function” as used herein refers to those disorders that in one way or another result from or in failure of the mitochondria, specialized compartments present in cells that are responsible for creating more than 90% of the energy needed by the body to sustain life and support growth. When mitochondrial function fails, less energy is generated within the cell. Cell injury and ultimately cell death follow. Such conditions include those that have neuromuscular disease symptoms (often referred to as “mitochondrial myopathy”), diabetes mellitus, multiple sclerosis, subacute sclerosing encephalopathy, dementia, myoneurogenic gastrointestinal encephalopathy, Parkinson's disease, Huntington disease, Amyotrophic Lateral Sclerosis (ALS), mental retardation, deafness and blindness, obesity, heart failure, stroke, lupus, and rheumatoid arthritis. Such conditions also include the relative ability to exercise. This includes, for example, recovery from immobilization of a body part or simply improving general exercise capacity.

The effects of mitochondrial disease can be quite varied, and mitochondrial diseases take on unique characteristics both because of the way the diseases are often inherited and because mitochondria are so critical to cell function. The severity of the specific defect may also be great or small. Some minor defects cause only “exercise intolerance”, with no serious illness or disability. Defects often affect the operation of the mitochondria and multiple tissues more severely, leading to multi-system diseases. Mitochondrial diseases as a rule are worse when the defective mitochondria are present in the muscles, cerebrum, or nerves as these cells use more energy than most in the body.

Although research is ongoing, treatment options are currently limited, though vitamins are frequently prescribed. Pyruvate has also been proposed recently as a treatment option. There remains a need in the art for prophylactic and therapeutic approaches for the treatment of these conditions.

### SUMMARY

It is an object of the invention to provide compositions and methods for prophylactic and/or therapeutic treatment of diseases and conditions related to apoptosis and cellular necrosis caused by ischemia. In various aspects described hereinafter, the present invention provides compositions and methods for treatment of acute coronary syndromes, including but not limited to myocardial infarction and angina; acute ischemic events in other organs and tissues, including but not limited to renal injury, renal ischemia and diseases of the aorta and its branches; injuries arising from medical interventions, including but not limited to coronary artery bypass grafting (CABG) procedures and aneurysm repair; and metabolic diseases, including but not limited to diabetes mellitus.

It is another object of the invention to provide compositions and methods for prophylactic and/or therapeutic treatment of conditions related to mitochondrial function. In various aspects described hereinafter, the present invention comprises administering one or more compounds selected from the group consisting of epicatechin, an epicatechin derivative, catechin, a catechin derivative, nicorandil, and a nicorandil derivative in an amount effective to stimulate mitochondrial function in cells. Stimulation of mitochondrial function in cells may comprise stimulation of one or more of mitochondrial respiration and mitochondrial biogenesis. The methods and compositions described herein can assist in prevention of impaired mitochondria biogenesis and thus prevention of the consequences of impaired mitochondrial biogenesis in various diseases and conditions, as well as provide for the active therapy of mitochondrial depletion that may have already occurred.

In a first aspect, the invention is directed to methods of treating an ischemic or ischemia/reperfusion (IR) condition in a subject. These methods comprise administering to a subject in need thereof a drug selected from the group consisting of epicatechin, derivatives thereof and pharmaceutically acceptable salts thereof, most preferably in combination with one or more drugs which have effects on ischemic disease.

In preferred embodiments, the subject is selected based on the occurrence of a myocardial infarction. Preferably the method reduces infarct size in the heart of the subject, and/or delays, attenuates or prevents adverse cardiac remodeling in the subject.

In other preferred embodiments, the subject is selected based on the occurrence of a renal injury. Preferably the method reduces the progression of the renal injury to renal failure. In still other preferred embodiments, the subject is selected based on the occurrence of a total coronary occlusion. Preferably the method reduces infarct size in the heart of the subject, and/or delays, attenuates or prevents adverse cardiac remodeling in the subject.

In still other preferred embodiments, the subject is selected based on the occurrence of acute myocardial ischemia (e.g., angina or AMI). Preferably the method reduces tolerance development to vasodilator drugs (e.g., nicroandil or a derivative thereof), and particularly to nitrate donor vasodilators such as nicorandil, nitroprusside and nitroglycerine, in the subject.

In yet other preferred embodiments, the subject is selected based on the occurrence of a stroke, an aortic aneurysm, atrial fibrillation.

In other preferred embodiments, the subject is selected based on the occurrence of medical intervention causing temporary acute ischemia, such as CABG surgery, aneurysm repair, angioplasty, or administration of a radiocontrast agent to the subject.

In certain embodiments of the present invention, epicatechin, or a derivative or pharmaceutically acceptable salt thereof, is administered to the subject together with one or more additional drugs useful in the treatment of ischemic or ischemia /reperfusion events. Exemplary additional drugs include one or more compounds independently selected from the group consisting of tetracycline antibiotics (e.g., doxycycline), glycoprotein IIb/IIIa inhibitors (e.g., eptifibatide, tirofiban, abciximab); ADP receptor/P2Y12 inhibitors (e.g., clopidogrel, ticlopidine, prasgurel); prostaglandin analogues (e.g., betaprost, iloprost, treprostinil); COX inhibitors (e.g., asprin, aloxiprin); other antiplatelet drugs (e.g., ditazole, cloricromen, dipyridamole, indobufen, picotamide, triflusal); anticoagulants (e.g., coumarins, 1,3-indandiones); heparins; direct factor Xa inhibitors; direct thrombin (II) inhibitors (e.g., bivalirudin); and vasodilators (e.g., fendoldopam, hydralazine, nesiritide, nicorandil, nicardipine, nitroglycerine, nitroprusside). This list is not meant to be limiting. In particularly preferred embodiments, epicatechin, or a derivative or pharmaceutically acceptable salt thereof, is administered together with one or more tetracycline antibiotics such as doxycycline.

While it is preferred that two or more drugs be “administered together” in the same pharmaceutical composition, the phrase as used herein is not intended to imply that this must be so. Rather, two or more pharmaceuticals are “administered together” if the T_{1/2} for the clearances of each pharmaceutical from the body overlaps at least partially with one another. For example, if a first pharmaceutical has a T_{1/2} for clearance of 1 hour and is administered at time=0, and a second pharmaceutical has a T_{1/2} for clearance of 1 hour and is administered at time=45 minutes, such pharmaceuticals are considered administered together. Conversely, if the second drug is administered at time=2 hours, such pharmaceuticals are not considered administered together.

Routes of administration for the pharmaceutical compositions of the present invention include parenteral and enteral routes. Preferred enteral routes of administration include delivery by mouth (oral), nasal, rectal, and vaginal routes. Preferred parenteral routes of administration include intravenous, intramuscular, subcutaneous, and intraperitoneal routes. When more than one pharmaceutical composition is being administered, each need not be administered by the same route. In particularly preferred embodiments, epicatechin, or a derivative or pharmaceutically acceptable salt thereof, is administered together intravenously with one or more tetracycline antibiotics such as doxycycline, most preferably in a single pharmaceutical composition.

Preferably, the pharmaceutical compositions of the present invention are administered in an “effective amount.” This term is defined hereinafter. Unless dictated otherwise, explicitly or otherwise, an “effective amount” is not limited to a minimal amount sufficient to ameliorate a condition, or to an amount that results in an optimal or a maximal amelioration of the condition. In the case when two or more pharmaceuticals are administered together, an effective amount of one such pharamaceutical may not be, in and of itself, be an effective amount, but may be an effective amount when used together with additional pharmaceuticals.

In certain embodiments, the pharmaceutical compositions of the present invention are administered within 48 hours of the onset of an ischemic or ischemia/reperfusion event or within 48 hours of presentation for medical treatment. Onset of an event may be identified by self-reporting of the subject, or by some objective measure of an event occurrence.

In the case of an ischemic event involving the heart, preferred objective measures include increases in one or more cardiac markers (e.g., CK-MB, myoglobin, cardiac troponin I, cardiac troponin T, B-type Natriuretic peptide, NT-proBNP, etc.); changes in serial ECG tracings; and angiographic results.

In the case of an ischemic event involving the kidneys, preferred objective measures include those defined by Bellomo et al., Crit Care. 8(4):R204-12, 2004, which is hereby incorporated by reference in its entirety,. This reference proposes the following classifications for stratifying acute kidney injury patients: “Risk”: serum creatinine increased 1.5 fold from baseline OR urine production of <0.5 ml/kg body weight for 6 hours; “Injury”: serum creatinine increased 2.0 fold from baseline OR urine production <0.5 ml/kg for 12 h; “Failure”: serum creatinine increased 3.0 fold from baseline OR creatinine >355 μmol/l (with a rise of >44) or urine output below 0.3 ml/kg for 24 h.

In preferred embodiments, the pharmaceutical compositions of the present invention are administered within 24 hours of the onset of an ischemic or ischemia/reperfusion event or patient presentation, more preferably within 12 hours, and most preferably within 6 hours.

In a related aspect, the present invention is directed to pharmaceutical compositions for treatment of an acute ischemic or ischemia /reperfusion (IR) event. This composition comprises an effective amount of epicatechin, or a derivative or pharmaceutically acceptable salt thereof, and one or more additional drugs useful in the treatment of ischemic or ischemia /reperfusion events. In particularly preferred embodiments, the pharmaceutical composition comprises epicatechin, or a derivative or pharmaceutically acceptable salt thereof, and one or more tetracycline antibiotics, most preferably doxycycline. Most preferably, the composition is formulated for intravenous delivery.

In another aspect, the present invention is directed to a method of enhancing or preserving migration, seeding, proliferation, differentiation and/or survival of stem cells in injured heart tissue of a subject comprising administering to a subject in need thereof a drug selected from the group consisting of epicatechin, derivatives thereof and pharmaceutically acceptable salts thereof, optionally administered together with one or more additional drugs useful in the treatment of ischemic or ischemia /reperfusion events.

In yet another aspect, the invention is directed to methods of treating metabolic disease in a subject. These methods comprise administering to a subject in need thereof a drug selected from the group consisting of epicatechin, derivatives thereof and pharmaceutically acceptable salts thereof. In preferred embodiments, the subject is selected based on the occurrence of diabetes. Preferably the method reduces blood glucose levels in the subject.

In another aspect, the present invention is related to certain derivatives of epicatechin These may find use in the methods described herein, or may be used in isolation as pharmaceutical compounds.

The term “epicatechin derivative” as used herein refers to any compound which retains the ring structure and 3R(-) stereochemistry of epicatechin itself, but which contains one or more substituent groups relative to epicatechin Certain naturally occurring epicatechin derivatives are known, such as (−)-epigallocatechin (EGC), (−)-epicatechin-3-gallate (ECG) and (−)-epigallocatechin-3-gallate (EGCG). The term also includes combination molecules or prodrugs which release epicatechin or a derivative thereof when administered to a subject. Such a combination molecule may include, for example, epicatechin and nicorandil joined by a hydrolysable linger group. Similarly, the term “catechin derivative” as used herein refers to any compound which retains the ring structure and 3R(₊) stereochemistry of catechin itself, but which contains one or more substituent groups relative to catechin

Preferred epicatechin derivatives have the following structure:

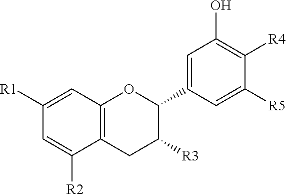

wherein
R1, R2, and R4 are each independently selected from the group consisting of —OH, —O—C₁₋₆ straight or branched chain alkyl, —O—C₁₋₁₂ arylalkyl, —C₁₋₆ straight or branched chain alkyl, and —C₁₋₁₂ arylalkyl, wherein each said straight or branched chain alkyl or arylalkyl comprises from 0-4 chain heteroatoms and optionally one or more substituents independently selected from the group consisting of halogen, trihalomethyl, —O—C₁₋₆ alkyl, —NO₂, —NH₂, —OH, —CH₂OH, —CONH₂, and —C(O)(OR6) where R6 is H or C₁₋₃ alkyl, provided that at least one of R1, R2, and R4 is not —OH, and provided that R4 is not —CH₃ or —O—CH₃ if R1 and R2 are each —OH;

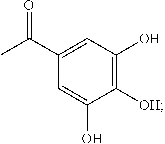

R3 is —OH or and
R5 is —H or —OH,
   or a pharmaceutically acceptable salt thereof.

In certain embodiments of such derivatives or pharmaceutically acceptable salts, two of R1, R2, and R4 are —OH. In still other embodiments, at least one of R1, R2, and R4 is —O—C₁₋₆ straight or branched chain alkyl.

Particularly preferred epicatechin derivatives include those having a structure selected from the groups consisting of

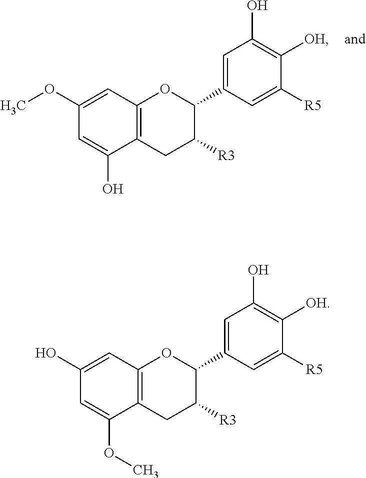

Such derivatives may be formulated as pharmaceutical compositions comprising a derivative or pharmaceutically acceptable salt described herein and a pharmaceutically acceptable excipient. These may be formulated for parenteral or enteral routes of administration.

In certain embodiments, such pharmaceutical compositions further comprise one or more compounds independently selected from the group consisting of tetracycline antibiotics, glycoprotein IIb/IIIa inhibitors, ADP receptor/P2Y12 inhibitors, prostaglandin analogues, COX inhibitors, antiplatelet drugs, anticoagulants, heparins, direct factor Xa inhibitors, direct thrombin (II) inhibitors, and vasodilators (e.g., nicroandil or a derivative thereof).

As noted above, it is another object of the invention to provide compositions and methods for prophylactic and/or therapeutic treatment of conditions related to mitochondrial function. In a first aspect, the present invention comprises administering one or more compounds selected from the group consisting of epicatechin, an epicatechin derivative, catechin, a catechin derivative, nicorandil, and a nicorandil derivative in an amount effective to stimulate mitochondrial function in cells.

Stimulation of mitochondrial function in cells may comprise stimulation of one or more of mitochondrial respiration and mitochondrial biogenesis. The methods and compositions described herein can assist in prevention of impaired mitochondria biogenesis and thus prevention of the consequences of impaired mitochondrial biogenesis in various diseases and conditions (both chronic and acute), as well as provide for the active therapy of mitochondrial depletion that may have already occurred.

In certain embodiments, the administration of compound(s) comprises administering at least 0.1 μM catechin, a catechin derivative, epicatechin or an epicatechin derivative to cells, at least 0.25 μM catechin, a catechin derivative, epicatechin or an epicatechin derivative, at least 0.5 μM catechin, a catechin derivative, epicatechin or an epicatechin derivative, and at least 1 μM catechin, a catechin derivative, epicatechin or an epicatechin derivative. In various embodiments, at least the desired concentration is maintained for at least 30 minutes, 1 hour, 3 hours, 6 hours, 12 hours, 24 hours, 48 hours, 72 hours, or more. In various other embodiments, at least the desired concentration is achieved at least once during each 12 hour period over at least 24 hours, 48 hours, 72 hours, 1 week, one month, or more; or at least once during each 24 hour period over at least 48 hours, 72 hours, 1 week, one month, or more. In order to maintain a desired concentration for a desired time, multiple doses of one or more compounds may be employed. The dosing interval may be determined based on the T1/2 for the clearances of each compound of interest from the body.

One or more compounds selected from the group consisting of epicatechin, an epicatechin derivative, catechin, a catechin derivative, nicorandil, and a nicorandil derivative may be delivered to an animal by a parenteral or enteral route in an amount effective to stimulate mitochondrial function in cells of said animal. Preferred enteral routes of administration include delivery by mouth (oral), nasal, rectal, and vaginal routes. Preferred parenteral routes of administration include intravenous, intramuscular, subcutaneous, and intraperitoneal routes. When more than one compound is being administered, each need not be administered by the same route.

Preferably, the compounds of the present invention are administered in an “effective amount.” This term is defined hereinafter. Unless dictated otherwise, explicitly or otherwise, an “effective amount” is not limited to a minimal amount sufficient to ameliorate a condition, or to an amount that results in an optimal or a maximal amelioration of the condition. In the case when two or more compounds are administered together, an effective amount of one such compound may not be, in and of itself, be an effective amount, but may be an effective amount when used together with additional compounds.

In those methods in which epicatechin, an epicatechin derivative, catechin, or a catechin derivative is delivered, it is preferred that the selected compound be at least 90% pure relative to other compounds selected from the group consisting of epicatechin, an epicatechin derivative, catechin, or a catechin derivative. For example, if the compound is epicatechin, it contains no more than 10% contamination with epicatechin derivatives, catechin, and catechin derivatives. More preferably the selected epicatechin, epicatechin derivative, catechin, or catechin derivative is at least 95% pure relative to other compounds selected from the group consisting of epicatechin, an epicatechin derivative, catechin, or a catechin derivative. It is noted that this does not exclude, however combination with nicorandil or a nicorandil derivative in substantial concentration. Thus in certain embodiments an epicatechin, an epicatechin derivative, catechin, or a catechin derivative is delivered in combination with nicorandil or a nicorandil derivative in the present methods. These are preferably provided in a single pharmaceutical composition.

An animal may be selected for administering one or more compounds selected from the group consisting of epicatechin, an epicatechin derivative, catechin, a catechin derivative, nicorandil, and a nicorandil derivative in an amount effective to stimulate mitochondrial function based on a diagnosis that said animal is suffering from or at immediate risk of suffering from one or more conditions involving decreased mitochondrial function. As noted above, such conditions can include inborn errors of mitochondrial metabolism, aging of the skin (e.g., due to light exposure), a nutritional or vitamin deficiency, mitochondrial myopathy, diabetes mellitus, insulin resistance, metabolic syndrome, Friedreich's ataxia, pulmonary hypertension, chronic kidney disease, acute kidney injury, hypertension, multiple sclerosis, subacute sclerosing encephalopathy, dementia or other conditions of impaired cognition related to aging, vascular disease, metabolic impairment or neurodegeneration (e.g., Alzheimer's disease), myoneurogenic gastrointestinal encephalopathy, Parkinson's disease, Huntington disease, Amyotrophic Lateral Sclerosis (ALS), mental retardation, deafness and blindness, obesity, heart failure, stroke, lupus, and rheumatoid arthritis.

An animal may be selected for administering one or more compounds selected from the group consisting of epicatechin, an epicatechin derivative, catechin, a catechin derivative, nicorandil, and a nicorandil derivative in an amount effective to stimulate mitochondrial function based on a desire to increase an ability to exercise. This includes, for example, recovery from immobilization of a body part or simply improving general exercise capacity. In addition an animal may be selected based on age, an activity state, or a nutritional state (e.g., subjects receiving total parenteral nutrition, infant formula, etc.) of said animal. This list is not meant to be limiting.

Thus, in various embodiments, the present invention provides a method for improving muscle structure or function; a method for improving mitochondrial effects associated with exercise; a method for enhancing the capacity for exercise in those limited by age, inactivity, diet, or any of the aforementioned diseases and conditions; a method for enhancing muscle health and function in response to exercise; a method for enhancing muscle health and function in the clinical setting of restricted capacity for exercise, whether due to injury, inactivity, obesity, or any of the aforementioned diseases and conditions; and/or a method to enhance recovery of muscles from vigorous activity or from injury associated with vigorous or sustained activity. In each case, the method comprises administering one or more compounds selected from the group consisting of epicatechin, an epicatechin derivative, catechin, a catechin derivative, nicorandil, and a nicorandil derivative in an amount effective to stimulate mitochondrial function in cells.

In preferred embodiments, the present invention comprises delivering catechin, a catechin derivative, epicatechin or an epicatechin derivative by an oral route in an amount effective to maintain a plasma concentration of at least 0.1 μM of said compound in said animal for at least 12 hours, 24 hours, 48 hours, 72 hours, or more. In various aspects, the method maintains a plasma concentration of at least 1 μM of said compound in said animal for at least 24 hours or more. In other preferred embodiments, the claimed invention comprises delivering catechin, a catechin derivative, epicatechin or an epicatechin derivative by an oral route in an amount effective to achieve a plasma concentration of at least 0.1 μM at least once during each 12 hour period over at least 24 hours, 48 hours, 72 hours, 1 week, one month, or more. In still other preferred embodiments, the claimed invention comprises delivering catechin, a catechin derivative, epicatechin or an epicatechin derivative by an oral route in an amount effective to achieve a plasma concentration of at least 0.1 μM at least once during or at least once during each 24 hour period over at least 48 hours, 72 hours, 1 week, one month, or more. In these embodiments, the method most preferably maintains or achieves a plasma concentration of at least 1 μM for the respective time periods recited above.

In related aspects, the present invention relates to treating a condition involving decreased mitochondrial function in an animal. These methods comprise delivering to the animal one or more compounds selected from the group consisting of epicatechin, an epicatechin derivative, catechin, a catechin derivative, nicorandil, and a nicorandil derivative to an animal by a parenteral or enteral route in an amount effective to stimulate mitochondrial function in cells of said animal.

In certain embodiments, the foregoing methods comprise delivering an effective amount of epicatechin or an epicatechin derivative. Preferred epicatechin derivatives have the following structure:

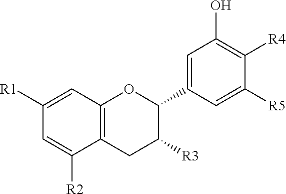

wherein
R1, R2, and R4 are each independently selected from the group consisting of —OH, —O—C₁₋₆ straight or branched chain alkyl, —O—C₁₋₁₂ arylalkyl, —C₁₋₆ straight or branched chain alkyl, and —C₁₋₁₂ arylalkyl, wherein each said straight or branched chain alkyl or arylalkyl comprises from 0-4 chain heteroatoms and optionally one or more substituents independently selected from the group consisting of halogen, trihalomethyl, —O—C₁₋₆ alkyl, —NO₂, —NH₂, —OH, —CH₂OH, —CONH₂, and —C(O)(OR6) where R6 is H or C₁₋₃ alkyl, provided that at least one of R1, R2, and R4 is not —OH, and provided that R4 is not —CH₃ or —O—CH₃ if R1 and R2 are each —OH;

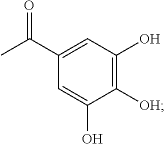

R3 is —OH or and
R5 is —H or —OH,
   or a pharmaceutically acceptable salt thereof.

In certain embodiments of such derivatives or pharmaceutically acceptable salts, two of R1, R2, and R4 are —OH. In still other embodiments, at least one of R1, R2, and R4 is —O—C1-6 straight or branched chain alkyl.

Particularly preferred epicatechin derivatives include those having a structure selected from the groups consisting of

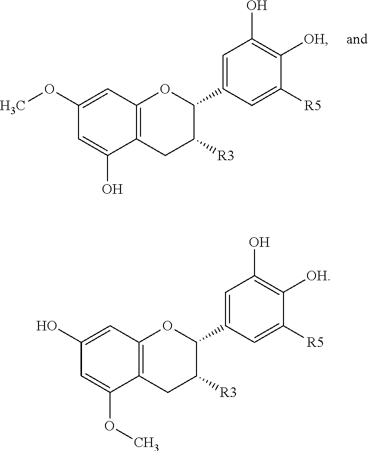

The term “nicorandil derivative” as used herein refers to any compound which retains the N-ethyl C-2 nitroxy moiety of N-[2-(Nitroxy)ethyl]-3-pyridinecarboxamide (nicorandil), but which contains one or more substituent groups relative to nicorandil. Examples include those disclosed in Boschi et al., Bioorg. Med. Chem. 8: 1727-32, 2000; and Satoh et al., Naunyn Schmiedebergs Arch Pharmacol. 344: 589-95, 1991. The term also includes combination molecules or prodrugs which release nicorandil or a derivative thereof when administered to a subject. Such a combination molecule may include, for example, epicatechin and nicorandil joined by a hydrolysable linger group.

The compounds and derivatives discussed above may be formulated as pharmaceutical compositions comprising a derivative or pharmaceutically acceptable salt described herein and a pharmaceutically acceptable excipient. These may be formulated for parenteral or enteral routes of administration. The compounds and derivatives discussed above may also be formulated as nutraceutical compositions as described hereinafter.

The details of one or more embodiments of the disclosure are set forth in the accompanying drawings and the description below. Other features, objects, and advantages of the disclosure will be apparent from the description and drawings, and from the claims.

### DESCRIPTION OF DRAWINGS

FIG. 1 depicts inhibition of mitochondrial pore opening by epicatechin and various derivitized forms thereof.

FIG. 2 depicts results from Example 3 showing I/R induced myocardial damage results in a ˜5 fold increase in arginase enzymatic activity (figure). Pretreatment (10 days) with (−)-epicatechin (1 mg/Kg) induced a significant decrease in arginase activity.

FIG. 3 depicts results from Example 4 showing that increases in intracellular Ca2⁺ do not correlate with increases in nitric oxide production in Epicatechin (EPI) treated Human Coronary Artery Endothelial Cells (HCAEC). Nitric oxide production and intracellular Ca2⁺ was separately measured in HCAEC treated with increasing concentrations of BK or EPI. In BK treated HCAEC, nitric oxide production mirrored increases in intracellular calcium at higher concentrations. HCAEC treated with 10 nM EPI and higher concentrations, showed a nitric oxide production greater than the increases of intracellular calcium.

FIG. 4 depicts results from Example 4 showing that EPI and BK treatment of HCAEC lead to intracellular Ca2⁺ concentration increases as measured by fluorescence. A. HCAEC treated with [1 mol/L] EPI and [1 mol/L] BK, displayed intracellular Ca2⁺ increases. Intracellular Ca2⁺ free HCAEC did not demonstrate increases in fluorescence despite EPI and BK treatment.

FIG. 5 depicts results from Example 4 showing that Nitric Oxide (NO) production was observed in Ca2+ free HCAEC treated with EPI. Approximately 25% NO production was seen in Ca2+ free HCAEC treated with [1 mol/L] EPI, in stark contrast to BK treatment, which was completely abrogated in the absence of intracellular Ca2+. [1 mol/L] BK treatment had a 2% of NO production, whereas [1 mol/L]EPI had 25%.

FIG. 6 depicts results from Example 4 showing that EPI activates endothelial nitric oxide synthatase (eNOS) through Ser-1177, 633 and 615 phosphorylation in absence of Ca2+. The relative phosphorylation of serine residues to total basal eNOS phosphorylation increased in [1 mol/L]EPI treated HCAEC. Phosphorylation of Ser-1177 increased by 100%, Ser-633 75% and Ser-615 by 65% versus the phosphorylation in the control. Changes in Thr-495 phosphorylation were not observed.

FIG. 7 depicts results from Example 4 showing that eNOS is activated by EPI in Ca2+ free HCAEC without disengaging from Caveolin-1 (Cav-1). Total protein from EPI or BK treated HCAEC was precipitated either Cav-1 or eNOS antibody. Western blots were performed in the immunoprecipitated phase against key eNOS residues, eNOS and Cav-1. In control HCAEC eNOS was not activated nor disengaged from Cav-1. BK treatment did not activate eNOS as observed by the phosphorylation status of Ser-1177, Ser-633 and Ser-615. Also, eNOS did not disengage from Cav-1.

FIG. 8 depicts results from Example 4 showing a Western blot of supernatant phase. Control, EPI and BK treated HCAEC, supernatant had negligible presence of eNOS residues, eNOS and Cav-1.

FIG. 9 depicts results from Example 4 showing that eNOS does not associate with Calmodulin-1 (CaM1) in Ca2⁺ free HCAEC treated with EPI or BK as well in the control. HCAEC were lysed and precipitated with eNOS antibody. The supernatant phase displayed only CaM1 expression but not eNOS.

FIGS. 10-15 depict results from Example 4 showing that eNOS is activated by EPI and remains in the cellular low-density phase corresponding to caveolae/lipid rafts in Ca2⁺ free HCAEC. Total protein extracts from HCAEC were arranged in a sucrose gradient. Sucrose gradient of 45, 35, interface and 5% were used for the detection of eNOS residues, eNOS, Cav-1, Transferrin Receptor (TfR) and Ganglioside M1 (GM1). FIG. 10: Control HCAEC in the presence of Ca2⁺ displayed an inactive eNOS, located in the low sucrose density fraction, along with Cav-1 and GM1. FIG. 11: BK treated HCAEC in the presence of regular Ca²⁺, had an activated eNOS located in the high sucrose density fraction as evidenced by the presence of TfR. FIG. 12: EPI treatment of HCAEC in the presence of regular Ca2⁺ activated eNOS and localized it to the high sucrose density fraction. FIG. 13: Control HCAEC free of Ca2⁺ had an inactive eNOS in the low sucrose density fraction. FIG. 14: BK was unable to activate and translocate eNOS to the high sucrose density fraction in Ca2⁺ free HCAEC. FIG. 15: EPI activated eNOS without translocation it to the high sucrose density fraction in Ca2⁺ free HCAEC.

FIG. 16 depicts the synthesis of 6ACA-EPI.

FIG. 17 depicts the observed decrease in % IA/AAR induced by the IV application of Dx-EPI.

FIG. 18 depicts the effect of epicatechin on the endogenous rate of respiration in C2C12 cells. OCR=oxygen consumption rate in pmoles O2/min/3×104 cells (mean±SD).

FIG. 19 depicts oxygen consumption rates (OCR) of endogenous, state 4 (resting), and uncoupler-stimulated respiration of C2C2 myoblasts treated with 0.1, 0.5 or 1 micromolar epicatechin for 48 hours. A. Rates over time with additions oligomycin to induce State 4 and FCCP to induce uncoupler stimulated respiration. B. Bar graphs of average rates from the same experiment. Data are mean±SD (n=3-4).

FIG. 20 depicts effects of epicatechin on the level of mitochondrial electron transport chain proteins. Western blots of C2C12 cells treated for 48 hours with epicatechin or catechin at 1 μM were probed with a cocktail of monoclonal antibodies toelectron transport chain proteins.

FIG. 21 depicts oxygen consumption rates (OCR) of endogenous, state 4 (resting), and uncoupler-stimulated respiration using primary cultures of human skeletal muscle myocytes resulting from nicroandil and epicatechin treatment.

FIG. 22 depicts comparative effects of nicorandil and epicatechin and the combination of these on oxygen consumption rates (OCR) of uncoupler-stimulated respiration using primary cultures of human skeletal muscle myocytes.

FIG. 23 depicts comparative effects of nicorandil and epicatechin on mitochondrial pore opening on oxygen consumption rates (OCR) of endogenous, state 4 (resting), and uncoupler-stimulated respiration using primary cultures of human skeletal muscle myocytes.

FIG. 24 depicts effects of epicatechin on mitochondrial pore opening.

FIGS. 25-27 depict the results of Example 10. FIG. 25 depicts effects of EPI, NICO and EPI+NICO (0.5 of individual doses) on mitochondrial swelling; FIG. 26 depicts an analysis of several combination of EPI+NICO indicating synergistic effects at very low concentrations; and FIG. 27 depicts isobolographic analysis of the combination of NICO (Y axis, [M]) and EPI (X axis, [M]) indicating a synergistic effect by the circle positioned off the line indicative of the additive effect.

FIG. 28 depicts effects of (−)-epicatechin (Epi) and/or nicorandil (Nico) treatment on infarct size using a rat model of myocardial ischemia-reperfusion (IR) injury.

### DETAILED DESCRIPTION

Unless specifically noted otherwise herein, the definitions of the terms used are standard definitions used in the art of pharmaceutical sciences. As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of or “consisting of.”

Unless defined otherwise, all technical and scientific terms used herein have the same meaning as commonly understood to one of ordinary skill in the art to which this disclosure belongs. Although any methods and reagents similar or equivalent to those described herein can be used in the practice of the disclosed methods and compositions, the exemplary methods and materials are now described.

All publications mentioned herein are incorporated herein by reference in full for the purpose of describing and disclosing the methodologies, which are described in the publications, which might be used in connection with the description herein. The publications discussed above and throughout the text are provided solely for their disclosure prior to the filing date of the disclosure. Nothing herein is to be construed as an admission that the inventors are not entitled to antedate such disclosure by virtue of prior disclosure.

Ischemia and reperfusion are physiologically different events and do not necessarily occur at the same time. As ischemia refers to deficiency of blood to a part typically due to a thrombus or embolus and reperfusion injury results when the obstruction or constriction is removed, it is possible and desirable to reduce the potential infarct size and adverse remodeling during the ischemia/reperfusion event. The disclosure provides methods and compositions useful for inhibiting ischemic and/or reperfusion injury comprising, for example, administering a epicatechin during the ischemia or alternatively after the ischemia, but before reperfusion has occurred, or alternatively after the ischemia and at the time of reperfusion. Disclosed herein are methods wherein epicatechin, a derivative thereof or a pharmaceutically acceptable salt thereof is administered during, prior to, or after an ischemia/reperfusion event.

Tissues deprived of blood and oxygen suffer ischemic necrosis or infarction, often resulting in permanent tissue damage. Cardiac ischemia is often termed “angina”, “heart disease”, or a “heart attack”, and cerebral ischemia is often termed a “stroke”. Both cardiac and cerebral ischemia result from decreased blood and oxygen flow which is often followed by some degree of brain damage, damage to heart tissue, or both. The decrease in blood flow and oxygenation may be the result of occlusion of arteries, rupture of vessels, developmental malformation, altered viscosity or other quality of blood, or physical traumas. Diabetes is a risk factor for ischemia. Accordingly, methods and compositions of the disclosure can be used to prevent or inhibit the risk of ischemia or inhibit and reduce the damage caused by ischemic injury in diabetic patients. This can include ischemia resulting in vision loss and ulcerations in addition to cardiac and cerebral ischemic injury.

Loss of blood flow to a particular vascular region is known as focal ischemia; loss of blood flow to the entire brain, global ischemia. When deprived of blood, and thus, oxygen and glucose, brain tissue may undergo ischemic necrosis or infarction. The metabolic events thought to underlie such cell degeneration and death include: energy failure through ATP depletion; cellular acidosis; glutamate release; calcium ion influx; stimulation of membrane phospholipid degradation and subsequent free-fatty-acid accumulation; and free radical generation.

Spinal cord injury is the most serious complication of spinal column trauma and also of operations on the aorta for treatment of thoracic and thoracoabdominal aneurysms (Kouchoukos, J. Thorac. Cardiovasc. Surg. 99:659-664, (1990)). As described in U.S. Pat. No. 5,648,331, the spinal cord is the organ most sensitive to ischemia during cross-clamping of the aorta, where the resultant injury may produce paraparesis or paraplegia. Spinal cord ischemia and paraplegia develop in approximately eleven percent (11%) of patients undergoing elective descending thoracic and thoracoabdominal aneurysm repair and nearly forty percent (40%) undergoing emergent repairs (Crawford, J. Vas. Surg. 3:389-402, (1986)).

Myocardial ischemia occurs when the heart muscle does not receive an adequate blood supply and is thus deprived of necessary levels of oxygen and nutrients. A common cause of myocardial ischemia is atherosclerosis, which causes blockages in the blood vessels (coronary arteries) that provide blood flow to the heart muscle. Congestive heart failure (CHF) can also result in myocardial infarction.

Ischemic events affecting the intestines play a major role of the mortality and morbidity or numerous patients. As described in U.S. Pat. No. 6,191,109, ischemic injury to the small intestine leads to mucosol destruction, bacterial translocation and perforation.

Age-related macular degeneration (AMD) is the leading cause of visual impairment and blindness in the United States and elsewhere among people 65 years or older. Oxidative damage to the retina may be involved in the pathogenesis of AMD.

Reactive oxygen species (ROS), also designated free radicals, include among other compounds singlet oxygen, the superoxide anion (O2-), nitric oxide (NO), and hydroxyl radicals. Mitochondria are particularly susceptible to damage included by ROS, as these are generated continuously by the mitochondrial respiratory chain. Production of ROS increases when cells experience a variety of stresses, including organ ischemia and reperfusion, ultraviolet light exposure and other forms of radiation. Reiter et al. (1998) Ann N.Y. Acad. Sci. 854:410-424; Saini et al. (1998) Res. Comm Mol. Pathol. Pharmacol. 101:259-268; Gebicki et al. (1999) Biochem. J. 338:629-636. ROS are also produced in response to cerebral ischemia, including that caused by stroke, traumatic head injury and spinal injury. In addition, when metabolism increases or a body is subjected to extreme exercise, the endogenous antioxidant systems are overwhelmed, and free radical damage can take place. Free radicals are reported to cause the tissue-damage associated with some toxins and unhealthful conditions, including toxin-induced liver injury. Obata (1997) J. Pharm. Pharmacol. 49:724-730; Brent et al. (1992) J. Toxicol. Clin. Toxicol. 31:173-196; Rizzo et al. (1994) Zentralbl. Veterinarmed. 41:81-90; Lecanu et al. (1998) Neuroreport 9:559-663.

The disclosure provides a method for treating and/or ameliorating the symptoms of an ischemic condition in a mammalian subject, comprising administering to the subject an effective amount of an epicatechin or epicatechin derivative alone or in combination with one or more drugs having an effect upon ischemic conditions. The disclosure also provides a method for treating and/or ameliorating the symptoms of an ischemic condition in a mammalian subject, comprising administering to the subject an effective amount of an epicatechin or epicatechin derivative alone or in combination with one or more drugs having an effect upon ischemic conditions, and by said administering, reducing tissue damage related to said ischemic condition. In some embodiments, the ischemic condition is selected from the group consisting of cerebral ischemia; intestinal ischemia; spinal cord ischemia; cardiovascular ischemia; myocardial ischemia associated with myocardial infarction; myocardial ischemia associated with CHF, ischemia associated with age-related macular degeneration (AME); liver ischemia; kidney/renal ischemia; dermal ischemia; vasoconstriction-induced tissue ischemia; penile ischemia as a consequence of priapism and erectile dysfunction; ischemia associated with thromboembolytic disease; ischemia associated with microvascular disease; and ischemia associated with diabetic ulcers, gangrenous conditions, post-trauma syndrome, cardiac arrest resuscitation, hypothermia, peripheral nerve damage or neuropathies. In some embodiments, the tissue ischemic condition is cerebral ischemia. In further embodiments, a subject is delivered epicatechin or an epicatechin derivative in a range of about 1 to about 1000 mg per kg body weight of said mammalian subject. In additional embodiments, a subject is delivered epicatechin or an epicatechin derivative in a range of about 1 to about 50 mg per kg body weight of said mammalian subject.

“Ischemia” or “ ischemic” or “an ischemic condition” refer to a medical event which is pathological in origin, or to a surgical intervention which is imposed on a subject, wherein circulation to a region of the tissue is impeded or blocked, either temporarily, as in vasospasm or transient ischemic attach (TIA) in cerebral ischemia or permanently, as in thrombolic occlusion in cerebral ischemia. The affected region is deprived of oxygen and nutrients as a consequence of the ischemic event. This deprivation leads to the injuries of infarction or in the region affected. The disclosure encompasses cerebral ischemia; intestinal ischemia; spinal cord ischemia; cardiovascular ischemia; ischemia associated with CHF, liver ischemia; kidney ischemia; dermal ischemia; vasoconstriction-induced tissue ischemia, such as a consequence of Raynaud's disorder; penile ischemia as a consequence of priapism; and ischemia associated with thromboembolytic disease; microvascular disease; such as for example diabetes and vasculitis; diabetic ulcers; gangrenous conditions; post-trauma syndrome; cardiac arrest resuscitation; and peripheral nerve damage and neuropathies; and other ischemias, including ischemia associated with ocular health concerns, such as for example, age-related macular degeneration (AMD). Ischemia occurs in the brain during, for example, a stroke, cardiac arrest, severe blood loss due to injury or internal hemorrhage and other similar conditions that disrupt normal blood flow. Ischemia occurs in myocardial tissue as a result of, for example, atherosclerosis and CHF. It may also occur after a trauma to the tissue since the pressure caused by edema presses against and flattens the arteries and veins inside the tissue, thereby reducing their ability to carry blood through the tissue. Cerebral ischemia may also occur as a result of macro-or micro-emboli, such as may occur subsequent to cardiopulmonary bypass surgery. Age-related macular degeneration may be associated with oxidative damage to the retina as a result of an ischemic condition. As used herein, a “non-cardiovascular” ischemic condition specifically excludes an ischemic condition of the cardio-pulmonary system or circulatory system. As used herein, a “non-cerebral” ischemic condition specifically excludes an ischemic condition of the brain.

“Cerebral Ischemia” or “cerebral ischemic” or “a cerebral ischemic condition” refer to a medical event which is pathological in origin, or to a surgical intervention which is imposed on a subject, wherein circulation to a region of the brain is impeded or blocked, either temporarily, as in vasospasm or transient ischemic attach (TIA) or permanently, as in thrombolic occlusion. The affected region is deprived of oxygen and nutrients as a consequence of the ischemic event. This deprivation leads to the injuries of infarction or in the region affected. Ischemia occurs in the brain during, for example, a thromboembolic stroke, hemorrhagic stroke, cerebral vasospasm, head trauma, cardiac arrest, severe blood loss due to injury or internal hemorrhage and other similar conditions that disrupt normal blood flow. It may also occur after a head trauma, since the pressure caused by edema presses against and flattens the arteries and veins inside the brain, thereby reducing their ability to carry blood through the brain. Cerebral ischemia may also occur as a result of macro-or micro-emboli, such as may occur subsequent to cardiopulmonary bypass surgery.

“Acute ischemia” or an “acute ischemic event” refers to an event having a sudden onset, as opposed to a chronic event which is ongoing.

In one aspect, methods of the disclosure relate to preventing neuronal damage in a mammalian subject at risk of developing injury due to a cerebral ischemic condition, e.g. for example, by an infarct in the brain. The methods of reducing neuronal damage relate to minimizing the extent and/or severity of injury in the brain associated with or due to a cerebral ischemic condition by ameliorating or reducing the injury that would otherwise occur. The disclosure provides prophylactic treatments for neuronal damage including cell death and/or presence of tissue edema and/or cognitive dysfunction and/or cerebral infarcts which may be due to ischemic, hypoxic/anoxic, or hemorrhagic events. The method is intended for a subject at risk of neuronal damage that is associated with, or results from, an acute or chronic medical condition. Such conditions might arise as a result of medical or surgical treatment planned for the subject (e.g., angioplasty) or as a result of an emergent medical condition such as a stroke or severe blood loss. Other conditions which place a subject at risk for neuronal damage associated with a cerebral ischemic condition include a genetic predisposition to stroke or a condition that is understood to increase the probability of incurring a cerebral infarct such as atherosclerosis, previous stroke or transient ischemic attacks, diabetes mellitus, hypertension, hypercholesterolemia, a history of smoking and may also include schizophrenia, epilepsy, neurodegenerative disorders, Alzheimer's disease and Huntington's disease. Diagnostic and/or pathological characterization of stroke victims has identified numerous additional medical conditions producing stroke that are widely known to practitioners of internal and neurological medicine.

In another aspect, methods of the disclosure relate to preventing myocardial damage in a mammalian subject at risk of developing injury due to a cardiovascular ischemic condition, e.g. for example, by a myocardial infarction or CHF. The methods of reducing myocardial damage relate to minimizing the extent and/or severity of injury in the heart associated with or due to a myocardial ischemic condition by ameliorating or reducing the injury that would otherwise occur. The disclosure provides prophylactic treatments for myocardial damage including cell death and/or presence of myocardial edema and/or myocardial infarcts which may be due to ischemic, hypoxic/anoxic, or hemorrhagic events. The method is intended for a subject at risk of myocardial damage that is associated with, or results from, an acute or chronic medical condition. Such conditions might arise as a result of medical or surgical treatment planned for the subject (e.g., angioplasty) or as a result of an emergent medical condition such as a myocardial infarction or severe blood loss. Other conditions which place a subject at risk for myocardial damage associated with a myocardial ischemic condition include a genetic predisposition to myocardial infarction or a condition that is understood to increase the probability of incurring a myocardial infarct such as atherosclerosis, CHF, previous myocardial infarction or transient ischemic attacks, diabetes mellitus, hypertension, hypercholesterolemia, and a history of smoking.

As used herein the phrase “adverse cardiac remodeling” refers to the changes in size, shape, and associated function of the heart after injury to the left and right ventricle and/or right and left atrium. The injury is typically due to acute myocardial infarction (such as, for example transmural or ST segment elevation infarction) or induced injury (such as for example, heart surgery), but may be from a number of causes that result in increased pressure or volume overload (forms of strain) on the heart. Cardiac remodeling includes hypertrophy, thinning of the myocardium, scar formation of the myocardium, atrophy of the myocardium, heart failure progression and combinations thereof. Chronic hypertension, Kawasaki's disease, congenital heart disease with intracardiac shunting, and valvular heart disease may lead to remodeling. Additionally remodeling may stem from coronary artery bypass surgery, cardiac transplant and application of a mechanical support device, such as a left ventricular assist device (LVAD).

As used herein “reduced myocardial infarct size” refers to a decrease in the size of a myocardial infarct in subjects treated with the compositions of the present invention compared to the size of a myocardial infarct in control subjects receiving no treatment. In the disclosed methods, “reducing” can refer to any one of a 5%, 10%, a 20%, a 30%, a 40%, or even a 50% decrease in myocardial infarct size. Alternately “reducing” can refer to any one of a 60%, 70% or 80% decrease in myocardial infarct size.

As is known to those of skill in the art, changes to the myocardium, particularly determination of the size of a myocardial infarct, can be made using imaging techniques such as echocardiography, cardiac MRI, cardiac CT, and cardiac nuclear scans. Additionally, elevation of one or more biomarkers, including troponin, CK-MB (creatine kinase mb), and CPK (creatine phosphokinase), is known to be indicative of dead or dying myocardium. There is also evidence that the biomarker BNP (B-type Naturetic Peptide) can be used as a marker for cardiac remodeling.

As used herein “favorable cardiac remodeling” refers to preservation of chamber size, shape, function and the prevention of ventricular wall thinning and scarring which occurs after injury to the heart.

As used herein “atrial fibrillation” and “atrial flutter” each refers to an arrhythmia where the atria do not beat effectively in coordination with the ventricle with often an accompanying decrease in cardiac output.

As used herein in reference to heart tissue “induced injury” refers to damaged myocardium, such as damage that results from heart surgery, including but not limited to, coronary artery bypass surgery, cardiac transplant and application of a mechanical support device, such as a left ventricular assist device (LVAD).

As used herein, an “ischemia/reperfusion event” includes, but is not limited to, myocardial ischemia, myocardial reperfusion, subarachnoid hemorrhage, ischemic strokes (including strokes resulting from cerebral thrombosis, cerebral embolism, and atrial fibrillation), hemorrhagic strokes (including strokes resulting from aneurysm and arteriovenous malformation), and transient ischemic attack, cardiac surgery where a heart lung machine is used such as coronary artery bypassing, and preservation of organs for transplant.

As used herein “ischemia/reperfusion injury” refers to damage to tissue caused when blood supply returns to the tissue after a period of ischemia. The absence of oxygen and nutrients from blood creates a condition in which the restoration of circulation results in inflammation and oxidative damage through the induction of oxidative stress rather than restoration of normal function.

Catechins are polyphenolic antioxidant found in plants. Catechins are flavonoids and, to be more specific, flavan-3-ols. Catechin and epicatechin are epimers, with (−)-epicatechin and (+)-catechin being the most common optical isomers found in nature.

Catechins constitute about 25% of the dry weight of fresh tea leaves although total the content varies widely depending on tea variety and growth conditions.

Catechins or Flavanols are found in teas and grapes and include, for example, monomeric flavan-3-ols catechin, epicatechin, gallocatechin, epigallocatechin, and epicatechin 3-O-gallate. Individuals at risk for ischemia/reperfusion events can decrease the risk of necrosis in future events by taking epicatechin, its pharmaceutically acceptable salt, or a derivative thereof prophylactically up to an indefinite period of time. It is also understood that many ischemia/reperfusion events have early warning symptoms preceding the actual event which can allow the subject to seek immediate treatment.

Even if there is injury caused by future ischemia/reperfusion events, it is contemplated that the prophylactic administration of the compositions of the present invention will reduce infarct size and adverse remodeling. For example, disclosed herein are methods of reducing the potential infarct size and adverse remodeling in a subject in need thereof comprising administering to the subject compositions of the present invention at least 30 minutes before a ischemia/reperfusion event. Disclosed herein are methods wherein a composition of the present invention is administered 15, 30 minutes, 1, 2, 6, 12, 24 hour(s), 2, 3 days, 1, or 2 weeks or any time point before the ischemia/reperfusion event.

Ischemia/reperfusion events can occur in subjects who are unaware of the impending infarction or ischemic event. In such individuals, there is a need to reduce the potential infarct size and adverse remodeling. Thus, the methods disclosed herein can be used to reduce the potential infarct size and adverse remodeling following the ischemia/reperfusion event.

In yet another embodiment, a composition of the present invention is administered prior to, following or concurrently with the administration of a tetracycline or derivative thereof. Exemplary tetracycline derivatives include, but are not limited to, chlortetracycline, oxytetracycline, demeclocycline, doxycycline, lymecycline, meclocycline, methacycline, minocycline, chlortetracycline, sancycline, chelocardin, apicycline; clomocycline, guamecycline, meglucycline, mepylcycline, penimepicycline, pipacycline, etamocycline, penimocycline and rolitetracycline. In addition, chemically modified tetracyclines can be used in the methods and compositions of the disclosure. Examples of chemically modified tetracyclines (CMTs) include:

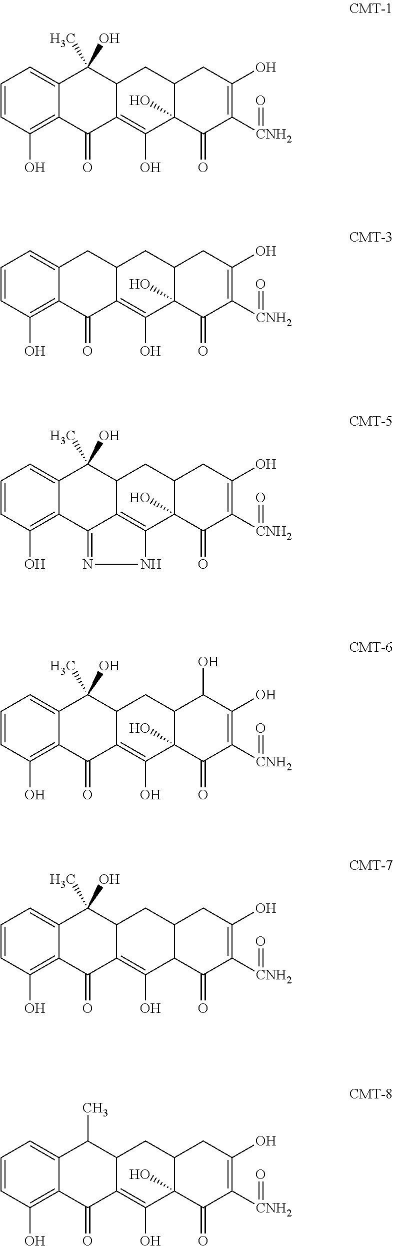

As described herein, the compositions of the present invention may comprise a reperfusion/thrombolytic agents (e.g., a tPA or other reperfusion agent). Exemplary thrombolytic agents include alteplase, tenecteplase, reteplase, streptase, abbokinase, pamiteplase, nateplase, desmoteplase, duteplase, monteplase, reteplase, lanoteplase, microplasmin, Bat-tPA, BB-10153, and any combination thereof. Exemplary NMDA receptor antagonists include 3-alpha-ol-5-beta-pregnan-20-one hemisuccinate (ABHS), ketamine, memantine, dextromethorphan, dextrorphan, and dextromethorphan hydrobromide.

Epicatechin or a derivative or salt thereof can be formulated as disclosed herein or its presence otherwise can be created or increased, in combination with other agents commonly used in cardiac patients including, but not limited to, ACE inhibitors, beta blockers, diuretics, thromobolytic agents, NMDA receptor antagonists, spin-trap agents and aspirin. In addition epicatechin can be formulated with other naturally occurring agents including, but not limited to, resveratrol and vitamin E. Epicatechin can also be formulated with other agents administered to healthy individuals including, but not limited to, protein, vitamins, minerals, antioxidants, and the like.

The present disclosure also provides a method for prophylaxis and/or treatment of, and/or ameliorating the symptoms of, a condition related to mitochondrial function in a mammalian subject, comprising administering to the subject an effective amount one or more compounds selected from the group consisting of epicatechin, an epicatechin derivative, nicorandil, and a nicorandil derivative.

Individuals at risk for a condition related to mitochondrial function can decrease the risk of necrosis in future events by taking epicatechin, catechin, nicorandil, or pharmaceutically acceptable salts, or derivatives thereof prophylactically up to an indefinite period of time. In the event that there is a present condition related to mitochondrial function, it is contemplated that the prophylactic administration of the compositions of the present invention will reduce symptoms from such condition.

Epicatechin, catechin, nicorandil, or derivatives or salts thereof can be formulated as disclosed herein or its presence otherwise can be created or increased, in combination with other agents including, but not limited to, ACE inhibitors, beta blockers, diuretics, thromobolytic agents, NMDA receptor antagonists, spin-trap agents and aspirin. In addition epicatechin can be formulated with other naturally occurring agents including, but not limited to, resveratrol and vitamin E. Epicatechin can also be formulated with other agents administered to healthy individuals including, but not limited to, protein, vitamins, minerals, antioxidants, and the like.

In one variation of any of the embodiments or aspects disclosed herein a drug selected from the group consisting of epicatechin, derivatives thereof and pharmaceutically acceptable salts thereof is administered. In another variation of any of the embodiments or aspects disclosed herein epicatechin or a pharmaceutically acceptable salt thereof is administered. The epicatechin, its derivative or its salt administered via the means disclosed herein can be in any variety of concentrations, combination with other elements or agents, temperatures or other states best suited for the targeted applications.

Compounds of the disclosure are administered orally in a total daily dose of about 0.1 mg/kg/dose to about 100 mg/kg/dose, alternately from about 0.3 mg/kg/dose to about 30 mg/kg/dose. In another embodiment the dose range is from about 0.5 to about 10 mg/kg/day. Alternately about 0.5 to about 1 mg/kg/day is administered. Generally between about 25 mg and about 1 gram per day can be administered; alternately between about 25 mg and about 200 mg can be administered. The use of time-release preparations to control the rate of release of the active ingredient may be preferred. The dose may be administered in as many divided doses as is convenient. Such rates are easily maintained when these compounds are intravenously administered as discussed below.

For the purposes of this disclosure, the compounds may be administered by a variety of means including orally, parenterally, by inhalation spray, topically, or rectally in formulations containing pharmaceutically acceptable carriers, adjuvants and vehicles. The term parenteral as used here includes but is not limited to subcutaneous, intravenous, intramuscular, intraarterial, intradermal, intrathecal and epidural injections with a variety of infusion techniques. Intraarterial and intravenous injection as used herein includes administration through catheters. Administration via intracoronary stents and intracoronary reservoirs is also contemplated. The term oral as used herein includes, but is not limited to sublingual and buccal. Oral administration includes fluid drinks, energy bars, as well as pill formulations.

Pharmaceutical compositions containing the active ingredient may be in any form suitable for the intended method of administration. When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents; such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient is mixed with an inert solid diluent, for example calcium phosphate or kaolin, or as soft gelatin capsules wherein the active ingredient is mixed with water or an oil medium, such as peanut oil, liquid paraffin or olive oil.

Aqueous suspensions of the disclosure contain the active materials in admixture with excipients suitable for the manufacture of aqueous-suspensions. Such excipients include a suspending agent, such as sodium carboxymethylcellulose, methylcellulose, hydroxypropyl methylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

Oil suspensions may be formulated by suspending the active ingredient in a vegetable oil, such as arachis oil, olive oil, sesame oil or coconut oil, or a mineral oil such as liquid paraffin. The oral suspensions may contain a thickening agent, such as beeswax, hard paraffin or cetyl alcohol. Sweetening agents, such as those set forth above, and flavoring agents may be added to provide a palatable oral preparation. These compositions may be preserved by the addition of an antioxidant such as ascorbic acid.

Dispersible powders and granules of the disclosure suitable for preparation of an aqueous suspension by the addition of water provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

The pharmaceutical compositions of the disclosure may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan monooleate, and condensation products of these partial esters with ethylene oxide, such as polyoxyethylene sorbitan monooleate. The emulsion may also contain sweetening and flavoring agents.

Syrups and elixirs may be formulated with sweetening agents, such as glycerol, sorbitol or sucrose. Such formulations may also contain a demulcent, a preservative, a flavoring or a coloring agent.

The pharmaceutical compositions of the disclosure may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

The amount of active ingredient that may be combined with the carrier material to produce a single dosage form will vary depending upon the host treated and the particular mode of administration. For example, a time-release formulation intended for oral administration to humans may contain 0.07 to 1 7 mmol (approximately 20 to 500 mg) of active material compounded with an appropriate and convenient amount of carrier material-which may vary from about 5 to about 95% of the total compositions. It is preferred that the pharmaceutical composition be prepared which provides easily measurable amounts for administration.

As noted above, formulations of the disclosure suitable for oral administration may be presented as discrete units such as capsules, cachets or tablets each containing a predetermined amount of the active ingredient, as a powder or granules; as a solution or a suspension in an aqueous or non-aqueous liquid, or as an oil-in-water liquid emulsion or a water-in-oil liquid emulsion. The active ingredient may also be administered as a bolus, electuary or paste.

A tablet may be made by compression or molding, optionally with one or more accessory ingredients. Compressed tablets may be prepared by compressing in a suitable machine the active ingredient in a free flowing form such as a powder or granules, optionally mixed with a binder (e.g., povidone, gelatin, hydroxypropyl ethyl cellulose), lubricant, inert diluent, preservative, disintegrant (e.g., sodium starch glycolate, cross-linked povidone, cross-linked sodium carboxymethyl cellulose) surface active or dispersing agent. Molded tablets may be made by molding in a suitable machine a mixture of the powdered compound moistened with an inert liquid diluent. The tablets may optionally be coated or scored and may be formulated so as to provide. slow or controlled release of the active ingredient therein using, for example, hydroxypropyl methylcellulose in varying proportions to provide the desired release profile. Tablets may optionally be provided with an enteric coating, to provide release in parts of the gut other than the stomach. This is particularly advantageous with the compounds of formula 1 when such compounds are susceptible to acid hydrolysis.

Formulations suitable for topical administration in the mouth include lozenges comprising the active ingredient in a flavored base, usually sucrose and acacia or tragacanth; pastilles comprising the active ingredient in an inert base such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

Formulations suitable for vaginal administration may be presented as pessaries, tampons, creams, gels, pastes, foams or spray formulations containing in addition to the active ingredient such carriers as are known in the art to be appropriate.

Formulations suitable for parenteral administration include aqueous and non-aqueous isotonic sterile injection solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Injection solutions and suspensions may be prepared from sterile powders, granules and tablets of the kind previously described.

As used herein, pharmaceutically acceptable salts include, but are not limited to: acetate, pyridine, ammonium, piperazine, diethylamine, nicotinamide, formic, urea, sodium, potassium, calcium, magnesium, zinc, lithium, cinnamic, methylamino, methanesulfonic, picric, tartaric, triethylamino, dimethylamino, and tris(hydoxymethyl)aminomethane. Additional pharmaceutically acceptable salts are known to those skilled in the art.

Analogously, derivatives of epicatechin are known to those of skill in the chemical arts. Such derivatives include, but are not limited to, epigallocatechin, epicatechin-3-gallate, and epigallocatechin-3-gallate.

As used herein, the term “an ischemic injury alleviating amount” or “effective amount” means the amount of a composition comprising a epicatechin or derivative or salt thereof useful for causing a diminution in tissue damage caused by ischemia. An effective amount to be administered systemically depends on the body weight of the subject. Typically, an effective amount to be administered systemically is about 0.1 mg/kg to about 100 mg/kg and depends upon a number of factors including, for example, the age and weight of the subject (e.g., a mammal such as a human), the precise condition requiring treatment and its severity, the route of administration, and will ultimately be at the discretion of the attendant physician or veterinarian.

The compositions of the present invention may also be formulated as neutraceutical compositions. The term “nutraceutical composition” as used herein refers to a food product, foodstuff, dietary supplement, nutritional supplement or a supplement composition for a food product or a foodstuff comprising exogenously added catechin and/or epicatechin Details on techniques for formulation and administration of such compositions may be found in Remington, The Science and Practice of Pharmacy 21st Edition (Mack Publishing Co., Easton, Pa.) and Nielloud and Marti-Mestres, Pharmaceutical Emulsions and Suspensions: 2nd Edition (Marcel Dekker, Inc, New York).

As used herein, the term food product refers to any food or feed suitable for consumption by humans or animals. The food product may be a prepared and packaged food (e.g., mayonnaise, salad dressing, bread, grain bar, beverage, etc.) or an animal feed (e.g., extruded and pelleted animal feed, coarse mixed feed or pet food composition). As used herein, the term foodstuff refers to any substance fit for human or animal consumption.

Food products or foodstuffs are for example beverages such as non-alcoholic and alcoholic drinks as well as liquid preparation to be added to drinking water and liquid food, non-alcoholic drinks are for instance soft drinks, sport drinks, fruit juices, such as for example orange juice, apple juice and grapefruit juice; lemonades, teas, near-water drinks and milk and other dairy drinks such as for example yoghurt drinks, and diet drinks. In another embodiment food products or foodstuffs refer to solid or semi-solid foods comprising the composition according to the invention. These forms can include, but are not limited to baked goods such as cakes and cookies, puddings, dairy products, confections, snack foods, or frozen confections or novelties (e.g., ice cream, milk shakes), prepared frozen meals, candy, snack products (e.g., chips), liquid food such as soups, spreads, sauces, salad dressings, prepared meat products, cheese, yogurt and any other fat or oil containing foods, and food ingredients (e.g., wheat flour).

Animal feed including pet food compositions advantageously include food intended to supply necessary dietary requirements, as well as treats (e.g., dog biscuits) or other food supplements. The animal feed comprising the composition according to the invention may be in the form of a dry composition (for example, kibble), semi-moist composition, wet composition, or any mixture thereof. Alternatively or additionally, the animal feed is a supplement, such as a gravy, drinking water, yogurt, powder, suspension, chew, treat (e.g., biscuits) or any other delivery form.

The term dietary supplement refers to a small amount of a compound for supplementation of a human or animal diet packaged in single or multiple dose units. Dietary supplements do not generally provide significant amounts of calories but may contain other micronutrients (e.g., vitamins or minerals). The term food products or foodstuffs also includes functional foods and prepared food products pre-packaged for human consumption.

The term nutritional supplement refers to a composition comprising a dietary supplement in combination with a source of calories. In some embodiments, nutritional supplements are meal replacements or supplements (e.g., nutrient or energy bars or nutrient beverages or concentrates).

Dietary supplements of the present invention may be delivered in any suitable format. In preferred embodiments, dietary supplements are formulated for oral delivery. The ingredients of the dietary supplement of this invention are contained in acceptable excipients and/or carriers for oral consumption. The actual form of the carrier, and thus, the dietary supplement itself, is not critical. The carrier may be a liquid, gel, gelcap, capsule, powder, solid tablet (coated or non-coated), tea, or the like. The dietary supplement is preferably in the form of a tablet or capsule and most preferably in the form of a hard (shell) capsule. Suitable excipient and/or carriers include maltodextrin, calcium carbonate, dicalcium phosphate, tricalcium phosphate, microcrystalline cellulose, dextrose, rice flour, magnesium stearate, stearic acid, croscarmellose sodium, sodium starch glycolate, crospovidone, sucrose, vegetable gums, lactose, methylcellulose, povidone, carboxymethylcellulose, corn starch, and the like (including mixtures thereof). Preferred carriers include calcium carbonate, magnesium stearate, maltodextrin, and mixtures thereof. The various ingredients and the excipient and/or carrier are mixed and formed into the desired form using conventional techniques. The tablet or capsule of the present invention may be coated with an enteric coating that dissolves at a pH of about 6.0 to 7.0. A suitable enteric coating that dissolves in the small intestine but not in the stomach is cellulose acetate phthalate.

In other embodiments, the dietary supplement is provided as a powder or liquid suitable for adding by the consumer to a food or beverage. For example, in some embodiments, the dietary supplement can be administered to an individual in the form of a powder, for instance to be used by mixing into a beverage, or by stirring into a semi-solid food such as a pudding, topping, sauce, puree, cooked cereal, or salad dressing, for instance, or by otherwise adding to a food or the dietary supplement e.g. enclosed in caps of food or beverage container for release immediately before consumption. The dietary supplement may comprise one or more inert ingredients, especially if it is desirable to limit the number of calories added to the diet by the dietary supplement. For example, the dietary supplement of the present invention may also contain optional ingredients including, for example, herbs, vitamins, minerals, enhancers, colorants, sweeteners, flavorants, inert ingredients, and the like.

In some embodiments, the dietary supplements further comprise vitamins and minerals including, but not limited to, calcium phosphate or acetate, tribasic; potassium phosphate, dibasic; magnesium sulfate or oxide; salt (sodium chloride); potassium chloride or acetate; ascorbic acid; ferric orthophosphate; niacinamide; zinc sulfate or oxide; calcium pantothenate; copper gluconate; riboflavin; beta-carotene; pyridoxine hydrochloride; thiamin mononitrate; folic acid; biotin; chromium chloride or picolonate; potassium iodide; sodium selenate; sodium molybdate; phylloquinone; vitamin D3; cyanocobalamin; sodium selenite; copper sulfate; vitamin A; vitamin C; inositol; potassium iodide. Suitable dosages for vitamins and minerals may be obtained, for example, by consulting the U.S. RDA guidelines.

In other embodiments, the present invention provides nutritional supplements (e.g., energy bars or meal replacement bars or beverages) comprising the composition according to the invention. The nutritional supplement may serve as meal or snack replacement and generally provide nutrient calories. Preferably, the nutritional supplements provide carbohydrates, proteins, and fats in balanced amounts. The nutritional supplement can further comprise carbohydrate, simple, medium chain length, or polysaccharides, or a combination thereof. A simple sugar can be chosen for desirable organoleptic properties. Uncooked cornstarch is one example of a complex carbohydrate. If it is desired that it should maintain its high molecular weight structure, it should be included only in food formulations or portions thereof which are not cooked or heat processed since the heat will break down the complex carbohydrate into simple carbohydrates, wherein simple carbohydrates are mono- or disaccharides. The nutritional supplement contains, in one embodiment, combinations of sources of carbohydrate of three levels of chain length (simple, medium and complex; e.g., sucrose, maltodextrins, and uncooked cornstarch).

Sources of protein to be incorporated into the nutritional supplement of the invention can be any suitable protein utilized in nutritional formulations and can include whey protein, whey protein concentrate, whey powder, egg, soy flour, soy milk soy protein, soy protein isolate, caseinate (e.g., sodium caseinate, sodium calcium caseinate, calcium caseinate, potassium caseinate), animal and vegetable protein and hydrolysates or mixtures thereof. When choosing a protein source, the biological value of the protein should be considered first, with the highest biological values being found in caseinate, whey, lactalbumin, egg albumin and whole egg proteins. In a preferred embodiment, the protein is a combination of whey protein concentrate and calcium caseinate. These proteins have high biological value; that is, they have a high proportion of the essential amino acids. See Modern Nutrition in Health and Disease, 8^{th} ed., Lea & Febiger, 1986, especially Volume 1, pages 30-32. The nutritional supplement can also contain other ingredients, such as one or a combination of other vitamins, minerals, antioxidants, fiber and other dietary supplements (e.g., protein, amino acids, choline, lecithin). Selection of one or several of these ingredients is a matter of formulation, design, consumer preferences and end-user. The amounts of these ingredients added to the dietary supplements of this invention are readily known to the skilled artisan. Guidance to such amounts can be provided by the U.S. RDA doses for children and adults. Further vitamins and minerals that can be added include, but are not limited to, calcium phosphate or acetate, tribasic; potassium phosphate, dibasic; magnesium sulfate or oxide; salt (sodium chloride); potassium chloride or acetate; ascorbic acid; ferric orthophosphate; niacinamide; zinc sulfate or oxide; calcium pantothenate; copper gluconate; riboflavin; beta-carotene; pyridoxine hydrochloride; thiamin mononitrate; folic acid; biotin; chromium chloride or picolonate; potassium iodide; sodium selenate; sodium molybdate; phylloquinone; vitamin D3 ; cyanocobalamin; sodium selenite; copper sulfate; vitamin A; vitamin C; inositol; potassium iodide.

The nutritional supplement can be provided in a variety of forms, and by a variety of production methods. In a preferred embodiment, to manufacture a food bar, the liquid ingredients are cooked; the dry ingredients are added with the liquid ingredients in a mixer and mixed until the dough phase is reached; the dough is put into an extruder, and extruded; the extruded dough is cut into appropriate lengths; and the product is cooled. The bars may contain other nutrients and fillers to enhance taste, in addition to the ingredients specifically listed herein.

It is understood by those of skill in the art that other ingredients can be added to those described herein, for example, fillers, emulsifiers, preservatives, etc. for the processing or manufacture of a nutritional supplement.

Additionally, flavors, coloring agents, spices, nuts and the like may be incorporated into the nutraceutical composition. Flavorings can be in the form of flavored extracts, volatile oils, chocolate flavorings, peanut butter flavoring, cookie crumbs, crisp rice, vanilla or any commercially available flavoring. Examples of useful flavoring include, but are not limited to, pure anise extract, imitation banana extract, imitation cherry extract, chocolate extract, pure lemon extract, pure orange extract, pure peppermint extract, imitation pineapple extract, imitation rum extract, imitation strawberry extract, or pure vanilla extract; or volatile oils, such as balm oil, bay oil, bergamot oil, cedarwood oil, walnut oil, cherry oil, cinnamon oil, clove oil, or peppermint oil; peanut butter, chocolate flavoring, vanilla cookie crumb, butterscotch or toffee. In one embodiment, the dietary supplement contains cocoa or chocolate.

Emulsifiers may be added for stability of the nutraceutical compositions. Examples of suitable emulsifiers include, but are not limited to, lecithin (e.g., from egg or soy), and/or mono- and di-glycerides. Other emulsifiers are readily apparent to the skilled artisan and selection of suitable emulsifier(s) will depend, in part, upon the formulation and final product. Preservatives may also be added to the nutritional supplement to extend product shelf life. Preferably, preservatives such as potassium sorbate, sodium sorbate, potassium benzoate, sodium benzoate or calcium disodium EDTA are used.

In addition to the carbohydrates described above, the nutraceutical composition can contain natural or artificial (preferably low calorie) sweeteners, e.g., saccharides, cyclamates, aspartamine, aspartame, acesulfame K, and/or sorbitol. Such artificial sweeteners can be desirable if the nutritional supplement is intended to be consumed by an overweight or obese individual, or an individual with type II diabetes who is prone to hyperglycemia.

Moreover, a multi-vitamin and mineral supplement may be added to the nutraceutical compositions of the present invention to obtain an adequate amount of an essential nutrient, which is missing in some diets. The multi-vitamin and mineral supplement may also be useful for disease prevention and protection against nutritional losses and deficiencies due to lifestyle patterns.

The dosage and ratios of catechin and/or epicatechin and additional components administered via a nutraceutical will vary depending upon known factors, such as the physiological characteristics of the particular composition and its mode and route of administration; the age, health and weight of the recipient; the nature and extent of the symptoms; the kind of concurrent treatment; the frequency of treatment; and the effect desired which can determined by the expert in the field with normal trials, or with the usual considerations regarding the formulation of a nutraceutical composition.

It will be understood, however, that the specific dose level for any particular patient will depend on a variety of factors including the activity of the specific compound employed, the age, body weight, general health, sex and diet of the individual being treated; the time and route of administration; the rate of excretion; other drugs which have previously been administered; and the severity of the particular disease undergoing therapy, as is well understood by those skilled in the art.

### EXAMPLES

### Example 1

Methylation of epicatechin produces at least 4 different products, mainly due to its 4 phenolic groups similar reactivity.

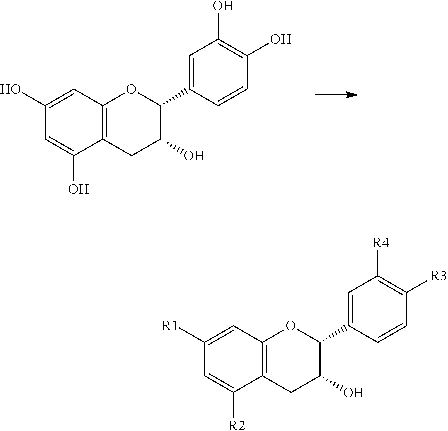

The general methylation reaction was adopted from Donovan, L. R., et al “Analysis of (+)catechin, (−)epicatechin and their 3′ and 4′O-methylated analogs, A comparison of sensitive methods” Journal of Chomatography B, 726 (1999):;277-283. Anhydrous K₂CO₃ (0.7 g), (CH₃)₂SO₄ (0.44 mL) and epicatechin (1g) were stirred into a mixture of H₂O (50 mL) and acetone (50 mL). Reaction was carried out during 3 hrs at room temperature in a sealed flask. Acetone was removed by rotary evaporation under reduced pressure. Reaction products were extracted (50 mL×2) with ethyl acetate. The products of this reaction, which include —O-methylated derivatives at each of R1, R2, R3, and R4, are separated by preparative chromatography and purified.

### Example 2

The prevention of the opening of mitochondrial pores when mitochondria are exposed to calcium overload is known to correlate to the protection of tissues from ischemic injury. The aperture of mitochondrial permeability transition pore (MPTP) can be evaluated through the measuring of mitochondrial swelling induced by the addition of calcium. (Bernardi P, Krauskopf A., Basso E., et al. The mitochondrial permeability transition from in vitro artifact to disease target. FEBS Journal 273:2077-99, 2006). Mitochondrial swelling is the result of water and electrolytes influx into the mitochondria through an calcium-induced MPTP opened. This phenomenon induce an increase in the light transmission at 535-540 nm (decrease on turbidity or decrease in absorbance at 535 nm) (Zoratti M and Szabo I. The mitochondrial permeability transition. Biochemic and Biophysic acta 1241:139-176, 1995).

Mitochondria were prepared from hearts of male Sprague-Dawley rats (250-300 g body wt.) and their protein content was determined The mitochondria were suspended in 70 mM-sucrose/210 mM-mannitol/10 mM-Tris/HCl, pH 7.2. Incubations were conducted at 25° C. and 1.0 mg of protein/ml in media which contained 10 mM succinate (Na+), 1.0 nmol/mg protein of rotenone, 3 mM Hepes (Na+), pH 7.4, plus mannitol/sucrose (3:1 mole ratio) to give a total osmotic strength of 300 mosm. Mitochondrial swelling was monitored at 540 nm in a spectrophotometer operated in the split beam mode. Swelling is recorded as a loss in light absorbance. The maximal value recorded for loss in light absorbance was normalized to =100%.

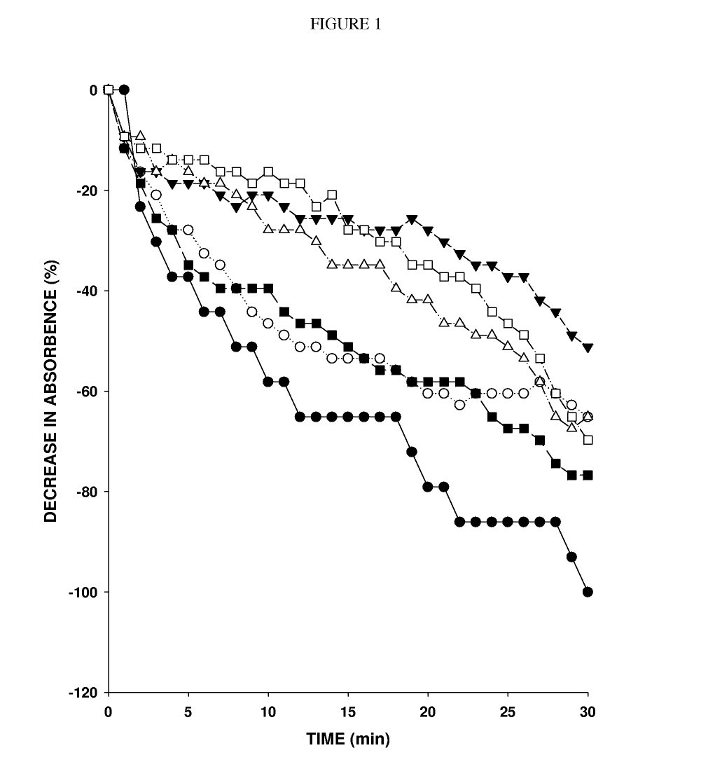

FIG. 1 depicts the effects of the various methylated epicatechin derivatives on opening of mitochondrial pores. The results obtained in the presence of 1 μM of each —O-methylated derivative (at each of R1, R2, R3, and R4 from Example 1) are shown as solid triangles, open triangles, open squares, and solid squares, respectively. For control comparisons, the results obtained using no compound (solid circles) and 1 μM underivitized epicatechein (open circles) are also shown. The following table provides a summary of these experiments.

TABLE 1

| | Inhibitory effect % | Inhibitory effect % |
|---|---|---|
| | (at 20 minutes) | (at 30 minutes) |
| No compound | — | — |
| Epicatechin | 27.5 | 35 |
| R1 —O—Me | 68.7 | 49 |
| R2 —O—Me | 48.75 | 35 |
| R3 —O—Me | 57.5 | 31 |
| R4 —O—Me | 27.5 | 24 |

From these results, it is observed that derivitization at the R1 position provides the greatest increase in potency, while derivitization at the R4 position reduces potency in this assay. However, the ability of the R4-O—CH3 derivative to stimulate NO production in human coronary artery endothelial cells (HCAEC) in culture was determined to be ˜46% greater in comparison to epicatechin

### Example 3

Endothelial dysfunction has been proposed as one of the mechanisms that contribute to microvascular injury and hypoperfusion after ischemia-reperfusion (I/R). The availability of L-arginine can be a rate-limiting factor for cellular nitric oxide (NO) production by nitric oxide synthases (NOS). Arginase, which shares L-arginine as a substrate with NOS, might compete for limited substrate and thereby regulate the activity of NOS in vascular endothelium. Increased arginase activity has been linked to low NO levels, and an inhibition of arginase activity has been reported to improve endothelium-dependent vasorelaxation. We have demonstrated that in rats (—)-epicatechin (EPI) can reduce the ischemia reperfusion (I/R) myocardial injury and is able to stimulate the synthesis of NO in human coronary endothelial cells in culture.

Others have shown that I/R inhibits NO-mediated dilation of coronary arterioles, by increasing the activity of the arginase (1). Elevated levels of arginase activity in cardiac tissue have been associated with clinical episodes of IR (2-3). We hypothesize that IR-induced increases in arginase activity can be prevented by epicatechin To test this hypothesis we examine the effects of EPI (1 mg/Kg) pretreatment (10 days) on myocardial arginase using a rat model of I/R injury.

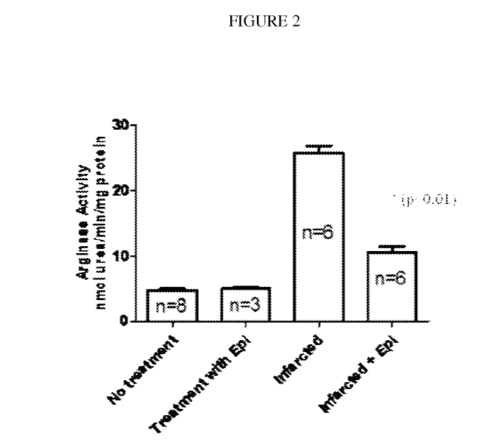

The general methods for the implementation of the rat myocardial I/R model are detailed in publications (4,5). The total time of myocardial ischemia was 45 min Hearts from 1) sham; 2) sham +EPI (10 days, 1 mg/Kg; gavage); 3) I/R and 4) I/R+EPI (10 days, 1 mg/Kg; gavage) were excised. Left ventricular tissue (0.120 g) was lysed with 0.5 ml of 25 mM Tris-HCl, 0.1% Triton X-100, 5 mM PMSF. The lysate was centrifuged (12000 rpm) 30 min at 4° C. and the precipitate eliminated. 25 μL of supernatant was added to 25 μL of buffer (25 mM Tris-HCl and 5 mM MnCl2 (pH 7.4). Arginase was then activated by heating the cell suspension for 10 min at 56° C. L-Arginine hydrolysis was conducted by incubating 25 μL of the activated lysate with 25 μL of 0.5 M L-arginine (pH 9.7) at 37° C. for 60 min The reaction was stopped with 400 μL an acidic mixture (H2SO4, H3PO4, and H2O; 1:3:7 v/v). Urea was measured at 545 nm after addition of 25 μL of 9% α-isonitrosopropiophenone (dissolved in 100% ethanol) and heating at 100° C. for 45 min to quantify arginase activity. Results indicate that I/R induced myocardial damage, results in a ˜5 fold increase in arginase enzymatic activity (FIG. 2). Pretreatment (10 days) with (−)-epicatechin (1 mg/Kg) induced a significant decrease in arginase activity (FIG. 2). I/R increases myocardial arginase activity in the left ventricle. Pretreatment with EPI suppresses this increase 48 h after IR. EPI induced cardioprotection may be related with increases in the availability of L-arginine to NOS via the inhibition of arginase.

### REFERENCES

1. O Schnorr, T Brossette, T Y. Momma, P Kleinbongard, C L. Keen, H Schroeter, H Sies. Cocoa flavanols lower vascular arginase activity in human endothelial cells in vitro and in erythrocytes in vivo. Archives of Biochemistry and Biophysics 476: 211-215, 2008

2. Morris S M Jr, Kepka-Lenhart D, Chen L C. Differential regulation of arginases and inducible nitric oxide synthase in murine macrophage cells. Am J Physiol Endocrinol Metab 275: E740-E747, 1998.

3. Xue G, Xiangbin X, SoBelmadani, Y Park, Z Tang, A. M. Feldman, W M. Chilian, C Zhang. TNF-α Contributes to Endothelial Dysfunction by Upregulating Arginase in Ischemia/Reperfusion Injury. Arterioscler Thromb Vasc Biol.; 27:1269-1275, 2007

4. Go Yamazaki K, D Romero-Perez, M Barraza-Hidalgo, M Cruz, M Rivas, B Cortez-Gomez, G Ceballos, and F Villarreal. Short- and long-term effects of (−)-epicatechin on myocardial ischemia-reperfusion injury. Am J Physiol Heart Circ Physiol 295: H761-H767, 2008

5. K G Yamazaki, P R Taub, M Barraza-Hidalgo, M M Rivas, A C Zambon, G Ceballos, F J Villarreal. Effects of (−)-epicatechin on myocardial infarct size and left ventricular remodeling following permanent coronary occlusion. J Am Coll Cardiol In Press, 2010

### Example 4

The NO production by eNOS has been extensively studied and it is well accepted that eNOS activation can be both, Ca²⁺-dependent and Ca²⁺-independent. Most humoral ligands, including BK, and acetylcholine stimulate eNOS activity by raising the level of intracellular ([Ca²⁺]ᵢ) which forms Ca^{2±}/calmodulin (Ca²⁺—CaM) complex (Yong Boo). On the other hand, mechanical forces such as fluid shear stress and stretching stimulate NO production by Ca²⁺-independent mechanisms (Yong Boo). Moreover, eNOS has been shown to be regulated by interactions with other positive and negative protein modulators such as caveolin-1 (Cav-1) and heat shock protein 90 (HSP90) (20, 41). In the basal state, the majority of eNOS appears to be bound to Cav-1 with its enzymatic activity repressed in the caveolae (27, 33). This tonic inhibition of eNOS can be released by displacing Cav-1 from eNOS with Ca²⁺/CaM binding in response to Ca²⁺-mobilizing agonists (27).

In addition to those modulators, phosphorylation of eNOS, at key regulatory sites, plays an important role in regulation of the enzyme activity in response to several physiological stimuli (3, 13, 17, 23, 35). It has been shown that phosphorylation of eNOS-Ser1177, Ser633 and Ser615 (human sequence) is associated with increased activity of the enzyme (19, 32), while phosphorylation of eNOS at Thr495 play an essential role in decrease enzyme activity (8, 23, 35, 36).

Interestingly in our previous work analyzing EPI-induced effects on human endothelial cells, we show that under pharmacological inhibition of intracellular pathways, that completely block bradykinin-induced effects on eNOS activity (i.e. PLC inhibition), EPI is still able, at least partially (˜30%), to induce NO production. These results suggested that EPI might be able to increase eNOS activity in a Ca2+ independent manner. We hypothesized that the flavonoid EPI activates eNOS independently of increases in intracellular calcium concentration and independently of its dissociation of caveola.

HCAEC and HCAEC growth medium were purchased from Cell Applications, Inc. EPI, protease and phosphatase inhibitors cocktails, caffeine, EGTA and cholera toxin subunit B (CTB) peroxidase conjugate were obtained from Sigma Chemicals. Phospho-eNOS Ser-1177, phospho-eNOS Ser-633, eNOS, Cav-1 primary antibodies, normal rabbit IgG control, and HRP-conjugated secondary antibodies from Cell Signaling Technology. Phospho-eNOS Thr-495, CaMI, phospho-CaMI, transferrin receptor primary antibodies were obtained from Santa Cruz Biotechnologies, phospho-eNOS Ser-615 antibody was from Millipore, Calcium green TM2 from Invitrogen. BK from EMD Biosciences. Nitrite/Nitrate Fluorometric Assay Kit from Cayman Chemical.

Cell Culture

HCAEC from 14, 40 and 60 year old healthy males were maintained in a humidified atmosphere at 37° C. with 5% CO2 and 95% O2 in HCAEC-growth medium. Treatments were typically applied to confluent cell cultures.

[Ca2+]ᵢ measurements.

HCAEC cultures were trypsinized and resuspended in HCAEC growth media. One ml of cell suspension (3×10⁵ cells/ml) was placed in each well of a 24 well dish plate and cells allowed to attach and settle for 24 hr. To maintain the cells in steady state of activity, 24 h before the experiments they were incubated with DMEM plus 0.5% FBS. Cells were incubated with M-199 without phenol red or FBS, and supplemented with 200 mM glutamine 6 h before experiments. Two experimental groups of HCAEC were generated; 1) regular calcium and 2) calcium deprived. Each group was subdivided for subsequent EPI or BK treatments. HCAEC were deprived of calcium by washing them (3×5 min) with Epilife media without Ca²⁺ or phenol red and supplemented with 1 mM EGTA and 1 mM caffeine. Cells were washed with either regular MOPS-Krebs-Henseleit solution (Krebs 1) composed of (in mM) 137 NaCl, 6 KCl, 1.8 CaCl₂, 1.2 NaH₂PO₄, 1.2 MgSO₄ 7H₂O, 5 dextrose, 2 sodium pyruvate, and 10 MOPS or with Ca²⁺ free-Krebs (Krebs 2). Cells were incubated 2 h at 37° C. with 500 μl of 3 μM Calcium Green TM2 diluted in their respective Krebs. Cells were washed and loaded with 500 μl Krebs1 or Krebs 2 (whichever applicable), 3×1 min Cells were allowed to settle for 1 h and then plate was inserted in a Synergy HT Fluorometer (BioTek). Either EPI or BK [0.1 nM-1 μM] were automatically applied to de cells plate to measure dose-response increases in intracellular calcium concentration [Ca²⁺]ᵢ at excitation and emission wavelengths of 503 nm 536 nm, respectively.

NO Measurements

NO levels were measured using a commercial kit and a fluorometer (FLx800 Bio-Tek Instruments INC) at excitation and emission wavelengths of 360 nm and 430 nm respectively. EPI was diluted in water and BK in DMSO (water or DMSO were used as vehicle for the control cells). EPI and BK-induced NO dose response curves were generated. For this experiments cells were treated with either [0.1 nmol/L-1 μmol/L] EPI and culture media samples were collected at 10 mM (peak time of NO response).

Immunoblotting

Cells grown on 10 cm dishes were homogenized in 50 μl lysis buffer (1% triton X-100, 20 mmol/L Tris, NaCl 140 mmol/L, mmol/L EDTA 2, 0.1% SDS) with protease and phosphatase inhibitor cocktails, supplemented with 1 mmol/L PMSF, 2 mmol/L Na₃VO₄ and 1 mmol/L NaF. Homogenates were passed through an insulin syringe 5×, sonicated for 30 min at 4° C. and centrifuged (12,000×g) 10 min at Total protein content was measured in the supernatant. A total of 40 μg of protein was loaded onto a 5 or 10% SDS-PAGE, electrotransferred, incubated 1 h in blocking solution (5% nonfat dry milk in TBS plus 0.1% Tween 20 [TBS-T]) followed by either a 3 h incubation at room temperature or overnight at 4° C. with primary antibodies. Primary antibodies were typically diluted 1:1000 or 1:2000 in TBS-T plus 5% bovine serum albumin Membranes were washed (3× for 5 min) in TBS-T and incubated 1 h at room temperature in the presence of HRP-conjugated secondary antibodies diluted 1:10,000 in blocking solution. Membranes were again washed 3× in TBS-T and the immunoblots developed using an ECL Plus detection kit (Amersham-GE). Band intensity was digitally quantified.

Immunoprecipitation.

Cells were lysed with 50 μl of non-denaturing extraction buffer (0.5%, Triton X-100, 50 mmol/L Tris-HCl ph 7.4; 0.15 mol/L NaCl; 0 5 mmol/L EDTA) and supplemented with protease and phosphatase inhibitors cocktail, plus 1 mmol/L PMSF, 2 mmol/L Na₃VO₄ and 1 mmol/L NaF. Homogenates were incubated on ice for 10 min and passed through an insulin syringe 5×. The homogenate was incubated on ice with shaking for 10 min and centrifuged (10 min) at 12,000×g at 4° C. A total of 0.5 mg protein was pre-cleared by adding 1 μg of normal rabbit IgG control and 20 μl prot-G-agarose with mixing for 30 min (4° C.) and subsequent centrifuging at 12,000×g for 10 min at 4° C. The supernatant was recovered and incubated at 4° C., under mild agitation with 3 μg of immunoprecipitating antibody (anti Cav-1 or anti CaMI for 3 h). Twenty μl of protein G-sepharose was added and the mixture was incubated at 4° C. for 3 h with shaking. The immunoprecipitation mixture was centrifuged at 12,000×g for 15 min at 4° C., and the supernatant recovered and stored at 4° C. The pellet was washed 3X with extraction buffer at 12,000×g for 15 min at 4° C. The immunoprecipitated proteins in the pellet and those remaining in the supernatant were applied to a 5 or 10% SDS-PAGE for immunoblotting. Co-immunoprecipitation was performed at least 3× with each immunoprecipitating antibody.

Detergent-Resistant Membrane (DRM) Isolation

Detergent-resistant membrane (lipid rafts and caveolae) isolation was performed as previously described (28, 33). Briefly: Approximately 4.5×10⁶ cells were lysed with 300 μl of cold TNE buffer (20 mM Tris, 140 mM NaCl, 2 mM EDTA) containing 0.05% Triton X-100, and protease and phosphatase inhibitors. Lysates were mixed with 375 μl of 80% sucrose in TNE-Triton X-100 buffer and transferred to ultracentrifuge tubes (catalog no. 347356; Beckman Coulter). Cell lysates, placed in 45% sucrose, were gently overlaid with 1 ml of 35% sucrose in TNE Triton X-100 buffer and this latter fraction was overlaid with 400 μl of 5% sucrose in TNE-Triton X-100 buffer. Samples were centrifuged at 4° C. for 16 h at 170,000×g in an Optima TLX ultracentrifuge using the TLS 55 rotor (Beckman Coulter). After centrifugation, eight 250 μl fractions were collected (top to bottom).

5 μl of each sucrose gradient fraction were placed onto a PVDF membrane. The drop was allowed to dry and the PVDF membrane was incubated 1 hr room temp in blocking solution. The PVDF membrane was subsequently incubated with 1:2000 CT-B-HRP dilution in blocking solution. The membrane was developed using an ECL Plus detection kit (Amersham-GE).

Data Analysis

A minimum of three experiments was performed (each in triplicate) unless otherwise noted. Statistical analysis was performed using t-test or ANOVA with significance noted at P<0.05.

Results

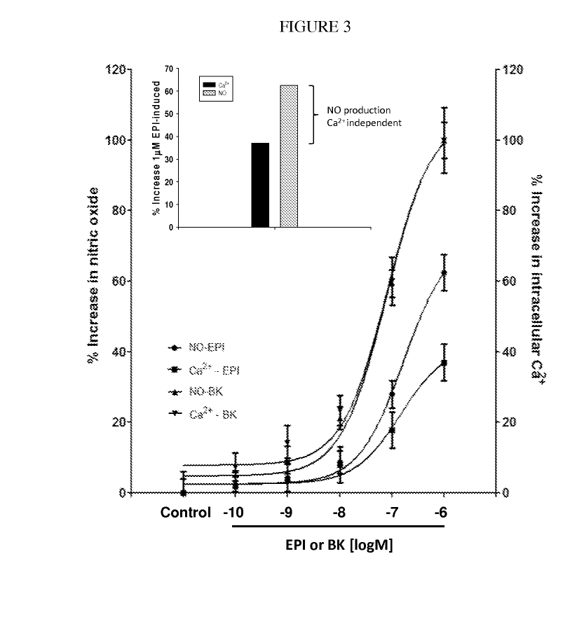

Based on existing literature documenting NO production in intracellular Ca²⁺ ([Ca²⁺i]) deprived endothelial cells, we proceeded to measure NO synthesis and increases in [Ca²⁺]ᵢ in HCAEC. (Fig.3). Cells were treated with increasing concentrations of EPI, and BK starting from 0 (control) to 1 μM. NO production and [Ca²⁺]ᵢ reached maximum levels at 1 μM in both; EPI and BK treatments. Interestingly increases in [Ca²⁺]ᵢ, were followed in parallel by increases in NO synthesis when the cells were treated with BK, however in cells treated with EPI the relationship between NO production and [Ca²⁺]ᵢ was not in parallel but NO production ratio is higher than [Ca²⁺]_{I} increases In other words, the ratio NO/[Ca²⁺]_{I} is higher in EPI-induced effects than in BK-induced effects, this is particularly evident from 10 nM-1 μM. This result, suggests that the activation of eNOS is partially Ca²⁺ independent in EPI treated HCAEC.

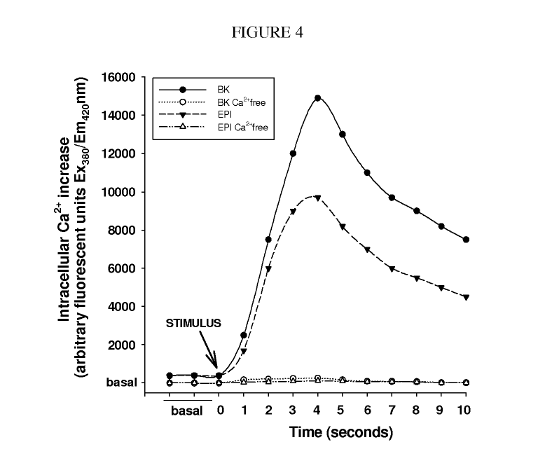

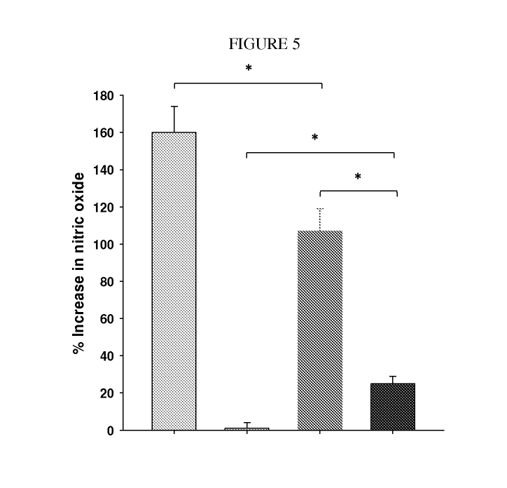

In endothelial cells, BK through activation of specific receptors, is a well known inducer of intracellular calcium kinetics, and therefore an eNOS activator, so it was interesting to assay the the possibility of EPI, that is also an eNOS activator, leads to an increase in [Ca²⁺]ᵢ in HCAEC since no specific receptors to EPI have been described. EPI as BK induces intracellular calcium kinetics; however EPI does it at lower levels than BK (FIG. 4). After depriving HCAEC of [Ca²⁺]ᵢ by caffeine and EGTA addition (3×, under Ca²⁺ free buffer), BK and EPI stimulation did not elicit [Ca^{2±}i] increase, indicating the efficacy of this technique in striping HCAECs of [Ca²⁺]ᵢ (FIG. 4). Once, we demonstrated the effectiveness of this technique to remove [Ca²⁺]ᵢ, we proceeded to measure NO production under various conditions. As expected, both BK and EPI lead to NO synthesis. Nevertheless, in Ca²⁺-free conditions only the BK induced NO synthesis was completely abrogated; whereas, EPI treated HCAEC despite being Ca²⁺-deprived still are capable of produce NO (approximately 30% of that synthesized under normal calcium conditions (FIG. 5).

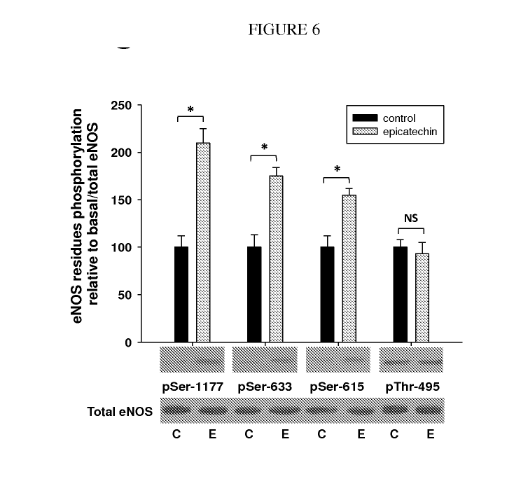

The phosphorylation status of Ser-1177, Ser-633, Ser-615 and Thr-495 is a measure of eNOS activity. Thus, in order to assess Query eNOS activation under Ca²⁺-free conditions, we measured the phosphorylation of these residues in EPI treated HCAECs. (FIG. 6) Changes in phosphorylation status were only observed in the residues Ser-1177, Ser-633 and Ser-615 (activation). These serines were significantly phosphorylated when compared to control HCAECs. Contrary to these results, the Thr-495 phosphorylation (inactivation) status was not altered, indicating its Ca²⁺ dependency. These results, suggest that eNOS activation under Ca²⁺-free conditions is mediated by changes in phosphorylation of Ser-1177, Ser-633, Ser-615 but not on Thr-495. Hence, the synthesis of NO observed under Ca²⁺-free conditions can be attributed to the phosphorylation of these residues.

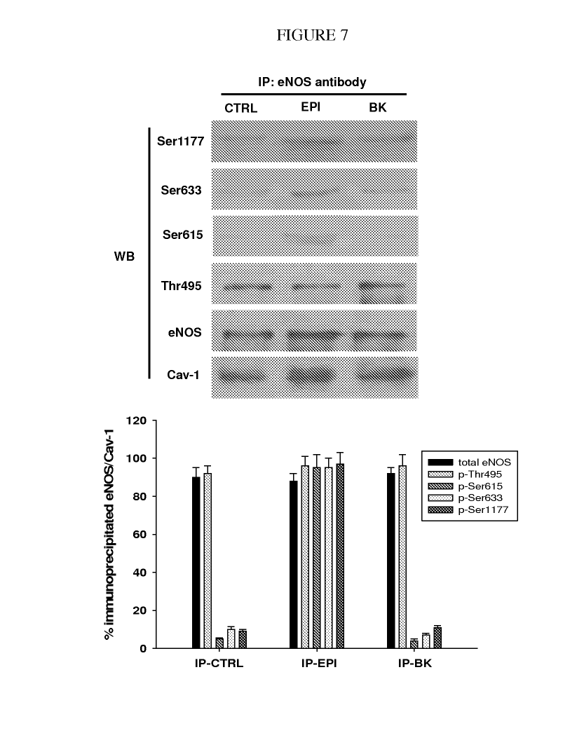

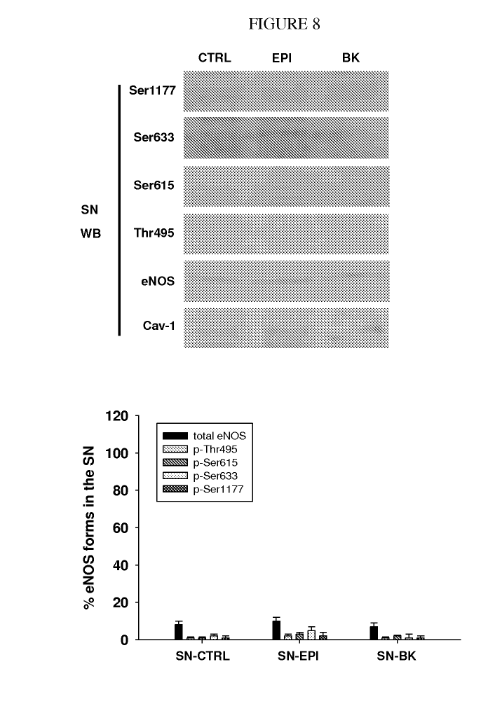

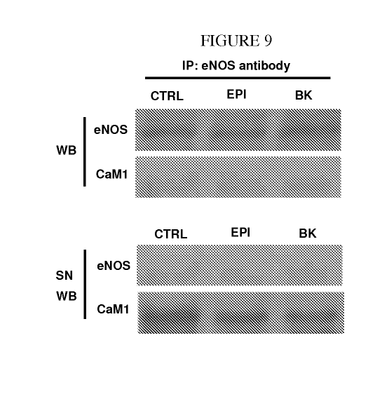

When Ca²⁺ is present, eNOS becomes activated and disengages from Caveolin-1 (Cav-1). Since we observed eNOS activation in EPI treated HCAECs in Ca²⁺-free conditions, we decided to explore whether it is also disengaged under this condition. Cav-1 was immunoprecipitated in; control, EPI and BK treated HCAECs under Ca²⁺-free conditions. The immunoprecipitated phase (IP) was then used for Western Blot analysis of eNOS residues and total eNOS as well Cav-1 (FIG. 7). eNOS in EPI and BK treated cells as well in the control cells, did not detached from de Cav-1, which suggest that Ca2+ is necessary to detach eNOS from the caveolae. In the absence of Ca²⁺ BK treated HCAEC resembled controlconditions because BK did not elicit phosphorylation changes in eNOS residues nor its dissociation from Cav-1 (FIG. 4A). In comparison, the IP phase of EPI treated HCAECs, showed significant phosphorylation of Ser-1177, Ser-633 and Ser-615 without dissociating from Cav-1, furthermore changes in Thr-495 phosphorylation were not observed, indicating that it is not required for eNOS activation. The WB for the supernatant (SN) phase of the IP don't show eNOS, neither phosphorylation of Ser-1177, Ser-633 and Ser-615, which indicates that eNOS still bound to caveolae after the treatment (FIG. 8). In addition we show the no association between eNOS and CaM after the cell treatment which indicates that CaM in not necessary to the eNOS activation in this condition (FIG. 9).

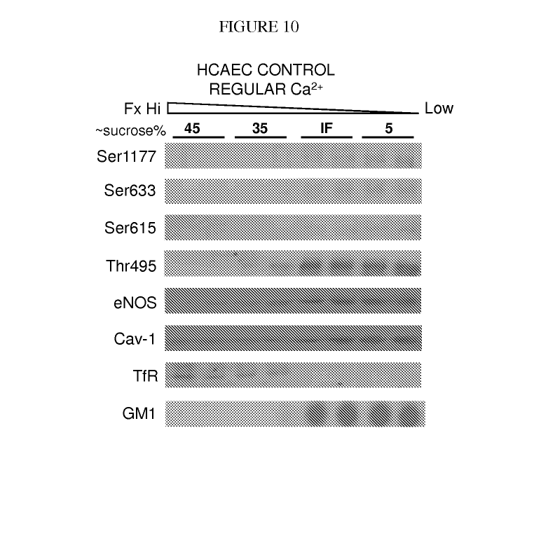

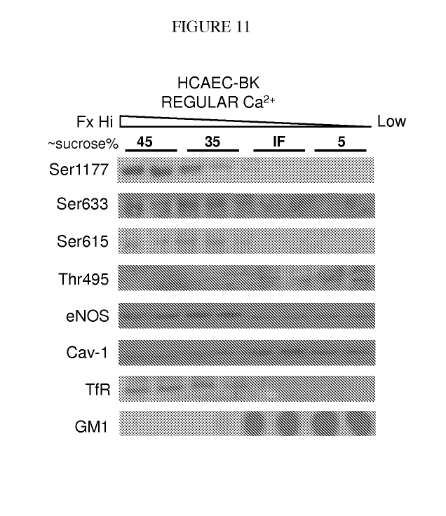

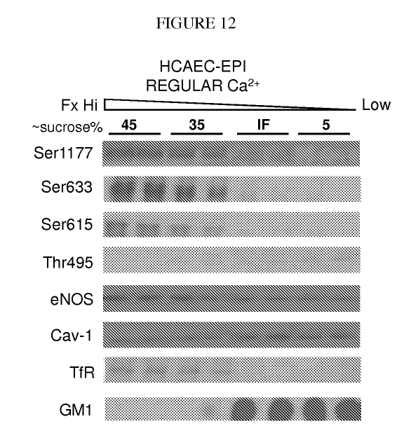

eNOS under physiological non-stimulated conditions is localized in membrane lipid rafts and caveolae. In order to further examine eNOS localization under Ca²⁺-free conditions in HCAEC, we created a subcellular fractionation on 45-35-interface (IF)-5% sucrose gradient. Each of these subcellular fractions were used to measure total eNOS, phosphorylation of Ser-1177, Ser-633, Ser-615 and Thr-495. In addition, antibodies to Cav-1 and the transferrin-receptor (TfR) were employed as controls, since; Cav-1 is found on low-density fractions whereas TfR shifts to high-density fractions. (FIG. 10) In control HCAECs, Ser-1177, Ser-633 and Ser-615 were not phosphorylated, while Thr-495 was phosphorylated, indicating eNOS inactivity. eNOS was found in the low-density sucrose fraction, along with Cav-1, while TfR was contained in the 45% sucrose fraction. (FIG. 11) The sucrose gradient of the BK-treated HCAECs, presented phosphorylation of Ser-1177, Ser-633 and Ser-615 and dephosphorylation of Thr-495, characteristics of eNOS activation. eNOS was mostly found in the 35% sucrose fraction, suggesting its translocation from low-density membrane lipids to the cytoplasm. (FIG. 12) Similar to BK, the sucrose gradient of EPI-treated HCAECs showed activation of eNOS, evidenced by the phosphorylation of Ser-1177, Ser-633 and Ser-615 and dephosphorylation of Thr-495. Furthermore, eNOS was localized in denser sucrose fractions, 45-35% along with TfR. Once we observed the activity and position of eNOS with respect to different subcellular fraction sucrose gradients, we repeated the experiments with the same stimuli with the exception of Ca^{2±}. In this new set of experiments, the cells were then Ca²⁺ deprived.

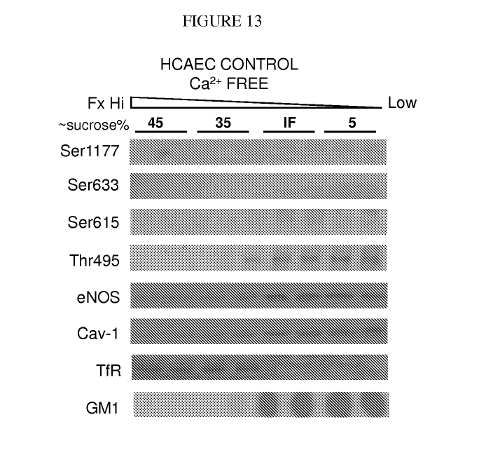

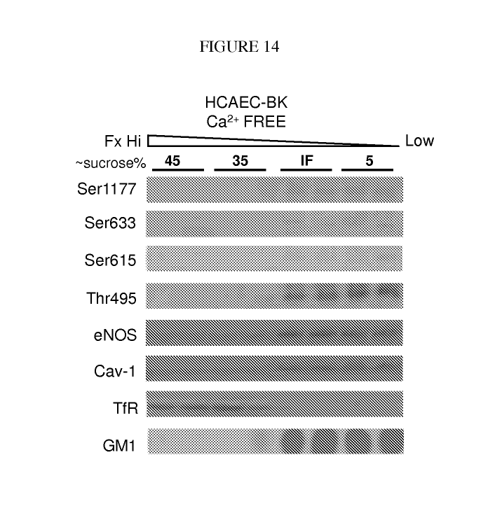

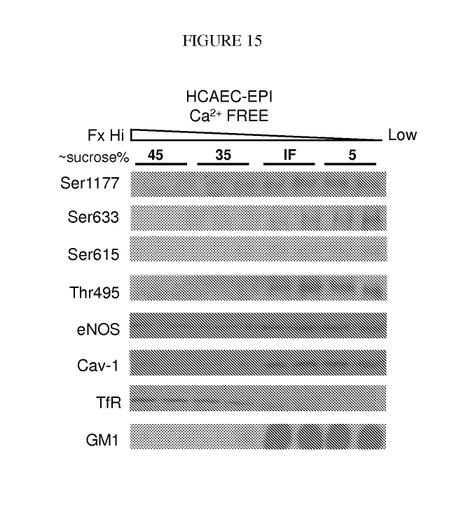

Control HCAECs exhibited an inactive eNOS localized to the low-density region of the sucrose gradient (FIG. 13). Ca²⁺-free HCAECs treated with BK did not express phosphorylation of Ser-1177, Ser-633 and Ser-615 or dephosphorylation of Thr-495, demonstrating eNOS inactivation. Moreover, eNOS did not translocate to denser sucrose fractions, and it was found in the 5% sucrose region along with Cav-1 (FIG. 14). This result is consistent with our previous experiments, were BK is shown to act through Ca²⁺. Treatment of HCAEC with EPI in Ca²⁺-free conditions, as seen in our previous experiments, led to the activation of eNOS. An important result from this experiment is that activated eNOS was localized in the low density sucrose fraction (IF-5%) and the Ser-1177, Ser-633 and Ser-615 residues were phosphorylated (FIG. 15). These results are indicate activation of eNOS without moving from the low-density region of membrane lipids.

EPI is able to activate of eNOS in a novel, calcium independent manner, this effect does not require the dissociation of the enzyme from caveola (cav-1) and is independent of calmodulin. EPI also increases eNOS protein levels by ˜40% and also induces mitochondrial biogenesis 48 h after treatment. Thus, unique effect may be partly responsible for the cardioprotective actions of EPI. EPI holds promise as an effective inducer of endothelial cell mitochondrial biogenesis. To the extent that this action is exerted, it can ameliorate adverse vascular effects of diseases such as DM in which endothelial mitochondria play a modulatory role.

### Example 5

To determine the effect that limiting the access of (−)-epicatechin (EPI), exclusively to the vascular lumen, has on infarct size using a rat model of myocardial ischemia-reperfusion (IR) injury. For this purpose a macromolecular (˜270 KDa) dextran-EPI (Dx-EPI) complex was synthesized. By preventing the free diffusion of EPI we thus, only evaluate EPI induced effects at the endothelium.

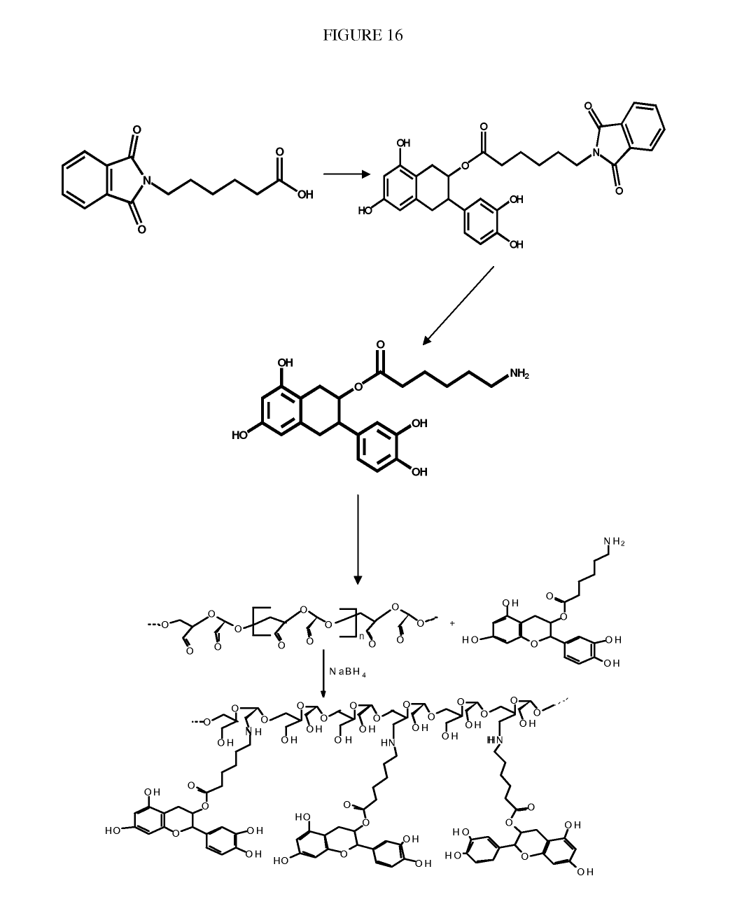

Synthesis of 6ACA-EPI was achieved through several chemical steps which are summarized in FIG. 16. Dx-EPI was synthesized using 6-aminocaproic acid (6ACA: 6 atoms) as a spacer arm between EPI and dextran thus, decreasing the steric effects of macromolecular dextran on EPI interacting molecules. The amino group of 6ACA was chemically protected to allow the reaction of its carbonyl with EPI to form an ester bound. The amino group was then deprotected in order to bind it to activated dextran. The Schiff base that was generated was then reduced to form a stable compound.

The general methods for the implementation of the rat myocardial IR model are detailed elsewhere. The total time of myocardial ischemia was 45 min Dx-EPI (3 mg/kg) was mixed in saline solution and given IV via the jugular vein. Control animals received dextran saline solution injections. Infarct size was examined 48 h after IR using established procedures.

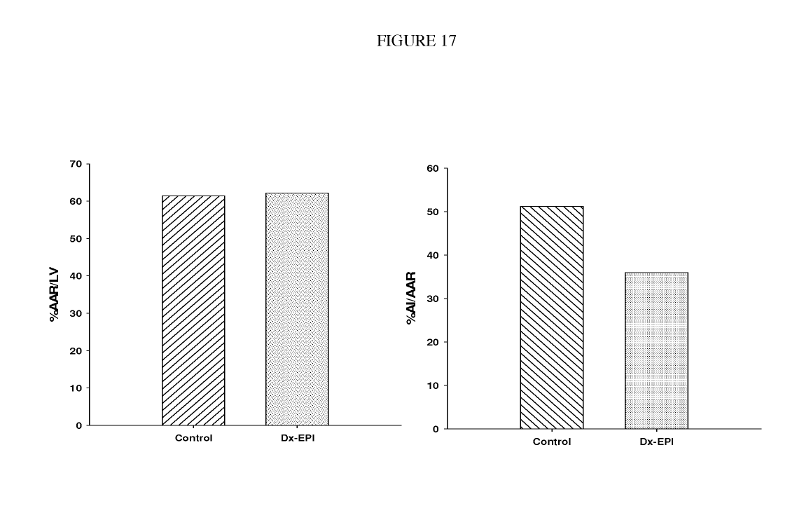

The resulting product has ˜0.254 mg of EPI per mg of macromolecular complex. In the IR studies we used 3 mg of complex/kg of rat (0.763 mg/kg in base of EPI content). Results from the IV administration of Dx-EPI are summarized in FIG. 17. Results suggest that interactions with endothelial cells (since Dx-EPI is essentially unable to freely diffuse) may be the major effectors of EPI induced cardioprotective effects. The content of applied EPI on macromolecular complex is ˜0.763 mg/kg this is a small quantity of EPI (compared with the 10 mg/kg of free EPI necessary to induce a significant cardioprotective effect.

### Example 6

Mitochondrial respiration is considered to be an overall marker of mitochondrial function, with increased oxygen consumption rate (OCR) thought to be a marker of improved mitochondrial function. The XF24 Extracellular Flux Analyzer (Seahorse Bioscience) uses fluorescence-based technology to simultaneously monitor O2 and pH levels in the medium over a cell monolayer in 24-well plates, which quantifies physiological changes in cellular energetics by measuring mitochondrial respiration and glycolysis. Measurement of both O2 consumption and pH enables a more comprehensive assessment of cellular energetics and the ability to determine the dynamic interplay between glycolytic ATP production in the cytoplasm and oxidative phosphorylation by the mitochondria.

Using the XF24 analyzer the effects of epicatechin at doses between 2.5-20 μM on rates of endogenous respiration in C2C12 mouse myoblasts were examined. Cultured cells were treated for 48 h with epicatechin, harvested with trypsin, and 30,000 cells were added per well to an XF24 plate coated with Cell-Tak (BD Biosciences) in DMEM containing 10 mM glucose, 10 mM pyruvate, and 2 mM glutamine. The plate was then centrifuged at 800×g for 5 min and transferred to the XF24 analyzer.

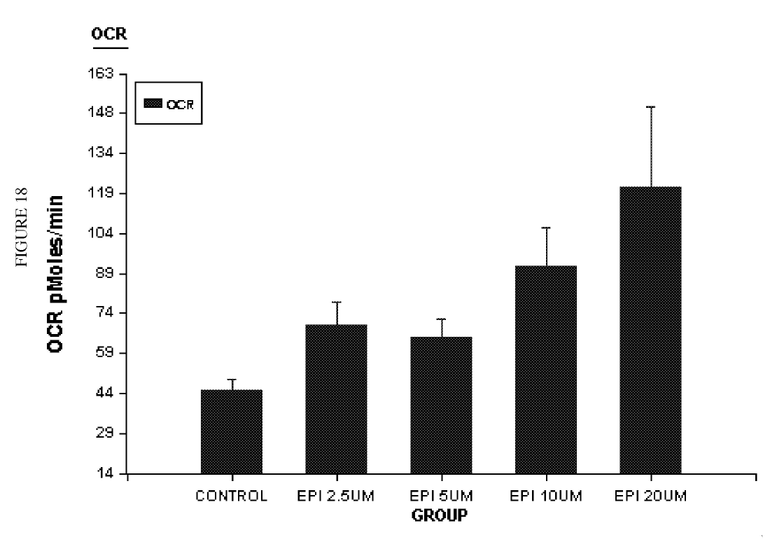

FIG. 18 demonstrates that epicatechin increases the endogenous rate of respiration in C2C12 cells in a dose-dependent manner. On electron microcopy there appears to be a statistically significant increase in cristae membrane where the oxidative phosphorylation pathway) composed of the electron transport chain and ATPase are located implying a greater capability for ATP generation with epicatechin treated cells as compared with control cells. This morphological change correlates with the improved mitochondrial function assessed via the Seahorse X24 analyzer.

### Example 7

Measured endogenous rates of respiration reflect the net balance between rates of energy utilization, energy production, and mitochondrial uncoupling. This was followed by induction of resting (State 4o) respiration with the addition of 1 uM oligomycin to inhibit ATP synthase. State 4 respiration is primarily determined by the rate of proton leak across the inner mitochondrial membrane. Maximal respiration was then assessed by the addition of 300 nM FCCP, a chemical uncoupler of oxidative phosphorylation. In intact cells, this rate reflects the maximal rates of substrate oxidation and electron transport chain activity. An increase in maximal rates can reflect changes in the regulation or expression level of oxidative enzymes, electron transport chain components, or total mitochondrial mass. The latter is influenced by the total mass of mitochondria in the cell. As a control, nonmitochondrial O₂ consumption was measured after the addition of 100 nM rotenone and 100 nM myxothiazol to completely block the respiratory chain.

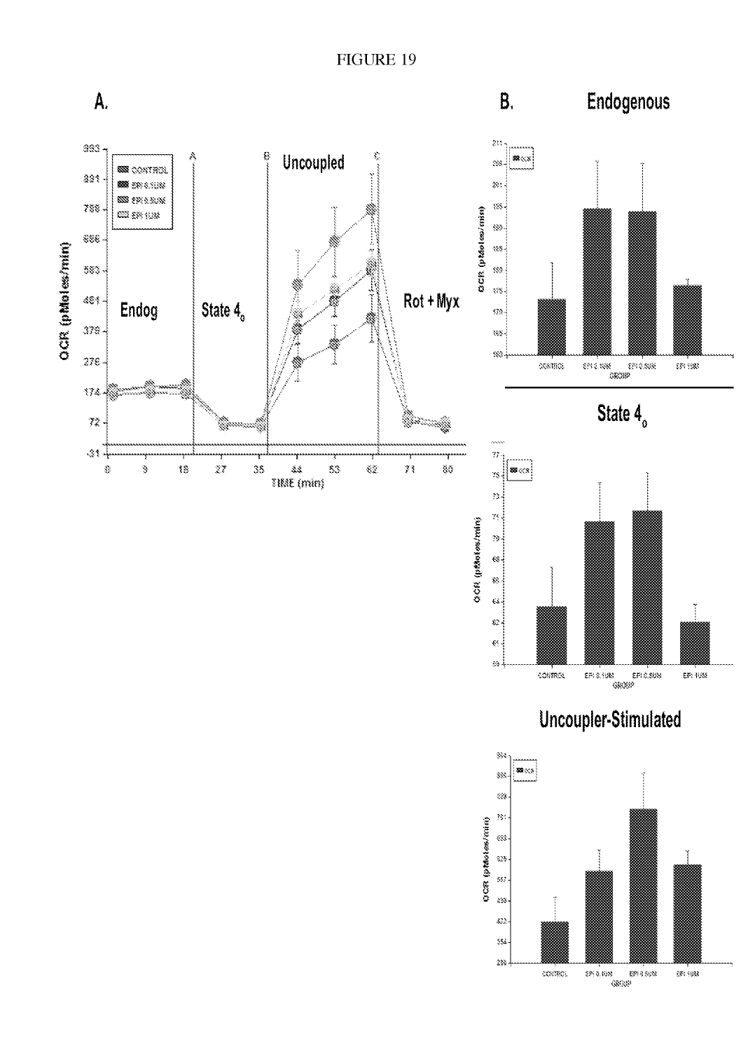

As demonstrated in FIG. 19, epicatechin at concentrations between 0.1 and 1.0 μM stimulated the rates of endogenous, state 4 (resting), and uncoupler-stimulated respiration. At concentrations above 5 μM, epicatechin was generally inhibitory to all rates of respiration (data not shown). These data suggest that epicatechin is inducing mild uncoupling, and also increasing either the maximal rates of substrate oxidation, the levels of rate-limiting components of electron transport, or the total mass of mitochondria in the cells.

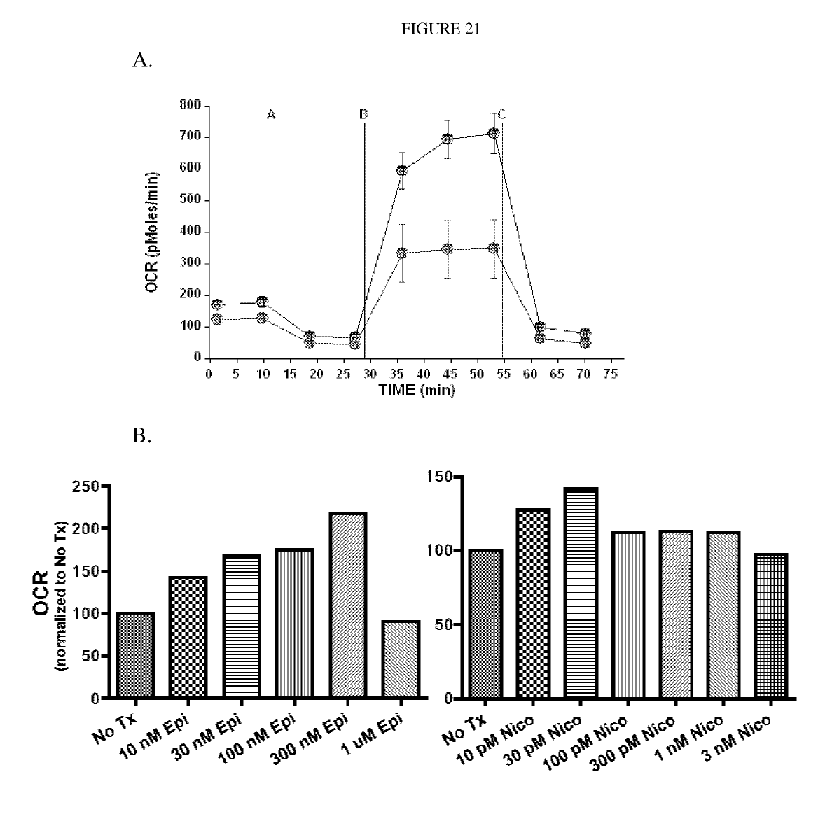

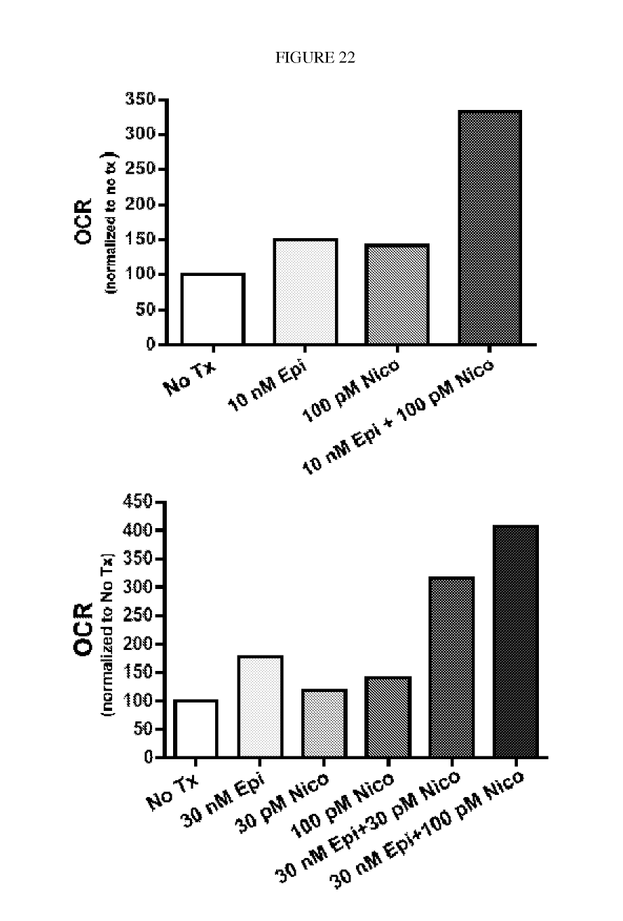

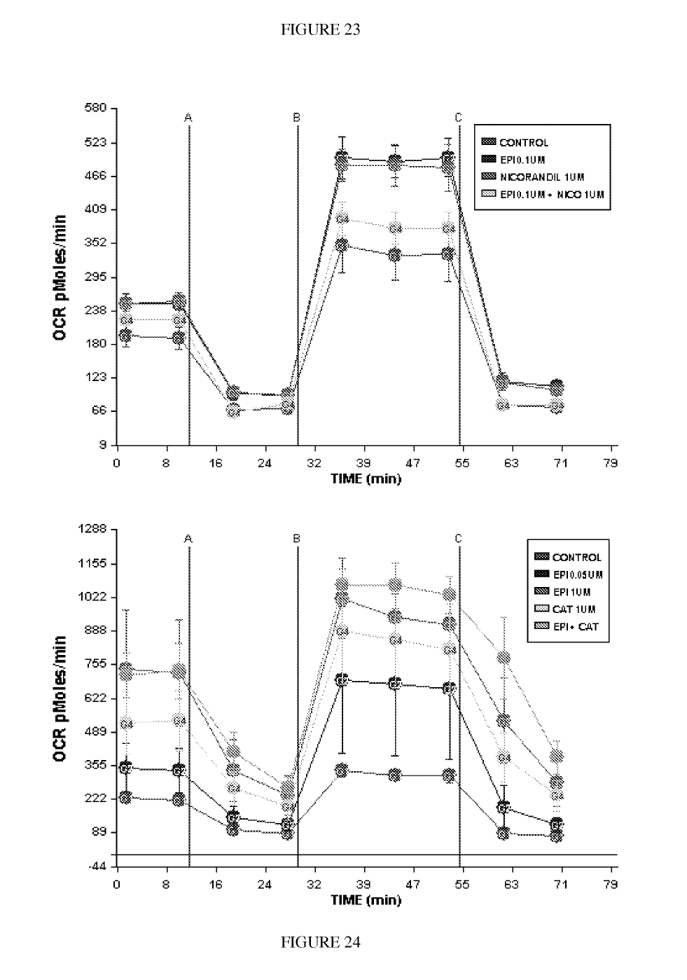

As shown in FIG. 21, these results were confirmed using primary cultures of human skeletal muscle myocytes (“HSKM cells”). Cells were plated at 30,000/well in XF24 plates and treated with the indicated concentration of epicatechin (top line in panel A; control in bottom line) in normal culture medium. Respiration of the intact cells was measured in unbuffered DMEM containing 10 mM glucose, 10 mM pyruvate, and 2 mM glutamine. In panel A, endogenous respiration was measured on HSKM cells, followed by state 4 (resting) respiration with the addition of 1 uM oligomycin (indicated as ‘A’), and then maximal rates were measured after the addition of 300 nM FCCP, a chemical uncoupler (indicated as ‘B’). Rotenone plus myxothiazol (100 nM each) was then added to assess non-mitochondrial oxygen consumption. In panel B, a dose response to epicatechin and nicorandil for 48 hours was performed with HSKM cells, and maximal rates of respiration were measured with addition of 300 nM FCCP. As shown in panel B and in FIG. 23, nicorandil and catechin are each active in stimulating mitochondrial function in this assay. As shown in FIG. 22, the effect of epicatechin and nicorandil together are synergistic.

### Example 8

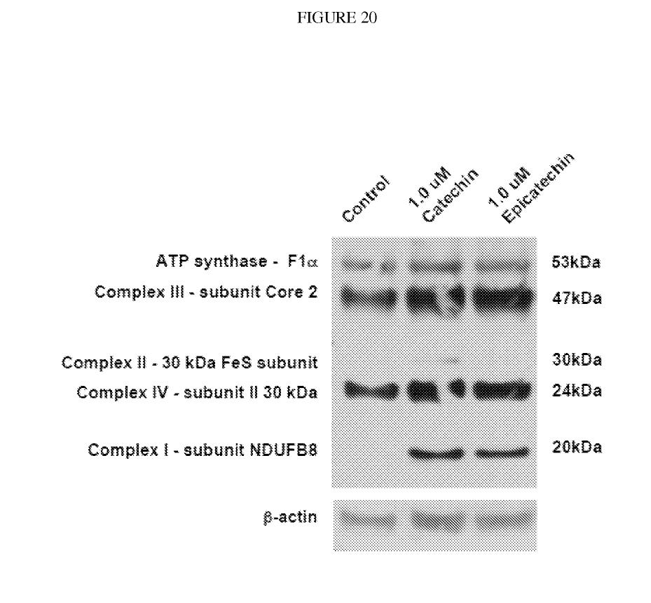

Western blots of cell lysates were used to assess levels of mitochondrial to determine if improved respiration is due to enhanced mitochondrial biogenesis. C2C12 cells treated for 48 hours with catechin or epicatechin at 1 μM were probed with a cocktail of monoclonal antibodies to electron transport chain proteins (MitoSciences MS601). As depicted in FIG. 20, epicatechin or catechin treatment has clearly increased the expression level of the 20 kDa subunit of complex I, and possibly induced slight increases in components of Complex III and IV.

### Example 9

The prevention of the opening of mitochondrial pores when mitochondria are exposed to calcium overload is known to correlate to the protection of tissues from ischemic injury. The aperture of mitochondrial permeability transition pore (MPTP) can be evaluated through the measuring of mitochondrial swelling induced by the addition of calcium. (Bernardi P, Krauskopf A., Basso E., et al. The mitochondrial permeability transition from in vitro artifact to disease target. FEBS Journal 273:2077-99, 2006). Mitochondrial swelling is the result of water and electrolytes influx into the mitochondria through an calcium-induced MPTP opened. This phenomenon induce an increase in the light transmission at 535-540 nm (decrease on turbidity or decrease in absorbance at 535 nm) (Zoratti M and Szabo I. The mitochondrial permeability transition. Biochemic and Biophysic acta 1241:139-176, 1995).

Mitochondria were prepared from hearts of male Sprague-Dawley rats (250-300 g body wt.) and their protein content was determined The mitochondria were suspended in 70 mM-sucrose/210 mM-mannitol/10 mM-Tris/HCl, pH 7.2. Incubations were conducted at 25° C. and 1.0 mg of protein/mL in media which contained 10 mM succinate (Na+), 1.0 nmol/mg protein of rotenone, 3 mM Hepes (Na+), pH 7.4, plus mannitol/sucrose (3:1 mole ratio) to give a total osmotic strength of 300 mosm. Mitochondrial swelling was monitored at 540 nm in a spectrophotometer operated in the split beam mode. Swelling is recorded as a loss in light absorbance. The maximal value recorded for loss in light absorbance was normalized to =100%.

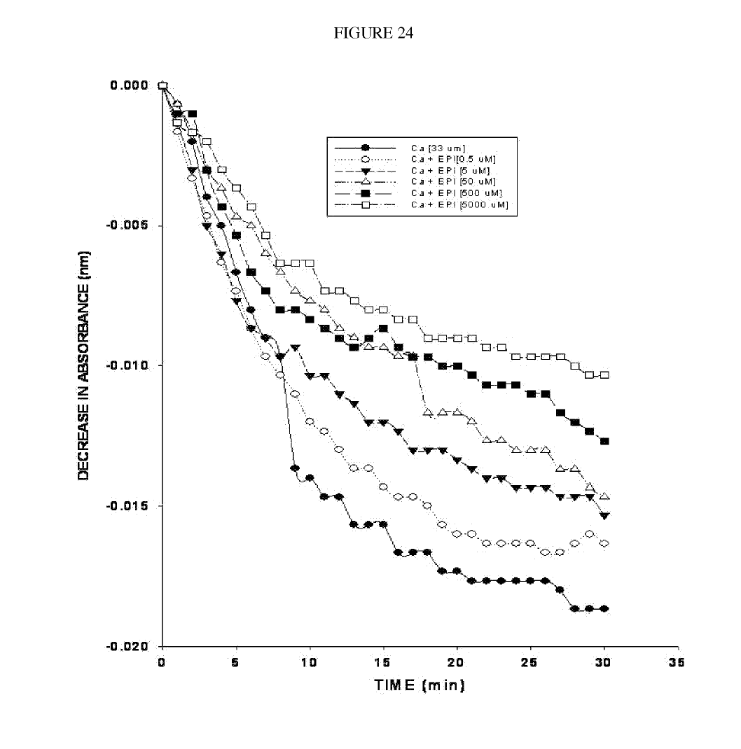

FIG. 24 depicts the inhibition of mitochondrial pore opening with increasing concentrations of epicatechin Catechins lacking the 3R(−) stereochemistry of epicatechin, while active in stimulating mitochondrial function, are not active in inhibiting mitochondrial pore opening. Thus, the 3R(−) catechins such as epicatechin exhibit an additional benefit relative to catechin and its derivatives in the claimed methods. It is also worth noting that epicatechin is superior to catechin in the ability to reduce infact size and in the ability to stimulate NO production in HCAEC cells. Thus, the combination of stereochemistry and substitution pattern can play an important role in the biological function of catechins

### Example 10

To determine the effect that (−)-epicatechin (EPI) and nicorandil (NICO) co-treatment has on mitochondrial swelling (damage) induced by high calcium, EPI or NICO, and EPI+NICO protective effects against calcium induced mitochondrial damage (swelling), were evaluated by monitoring changes in optical density (OD, light absorbance): Hearts from male rats were excised and weighed. Left ventricles were homogenized (0.1 g/mL) in solution A (Sucrose 2M, EDTA 0.01M, Hepes 0.5M: pH=7.4), centrifuged 10 min (800×g), 4° C., the supernatant was centrifuged 10 min (8000×g), 4° C. and the pellet was re-suspended in solution B (Sucrose 2M, EDTA 0.01M, Tris 0.5M-H2PO4-50 mM: pH=7.4) and centrifuged 10 min (10000×g), 4° C. Pellet was re-suspended in 10 mL of solution C (Sucrose 2M, EDTA 0.01M, Tris 0.5M-H2PO4-50 mM, Succinate 1M: pH=7.4. 33 μM of CaCl₂ was then, added in order to induce mitochondrial dammage (swelling measured through absorbace changes at 535 nm, monitored continuosly during 30 min

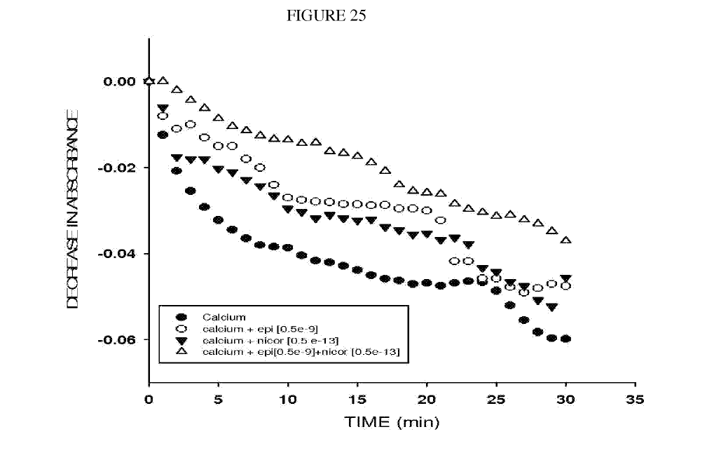

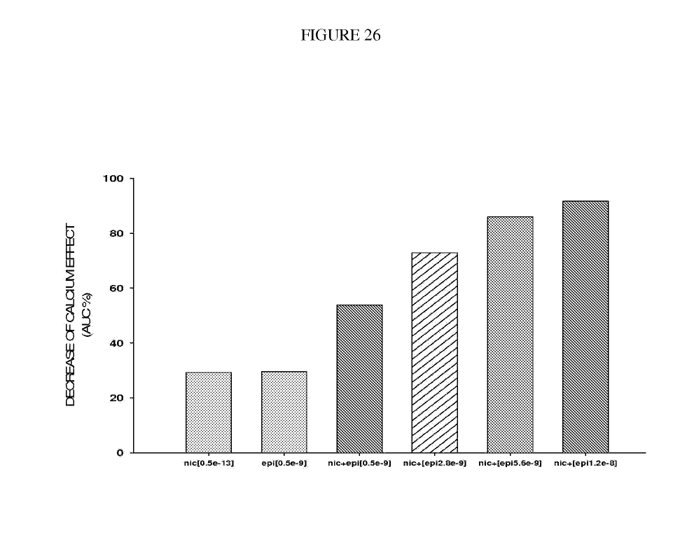

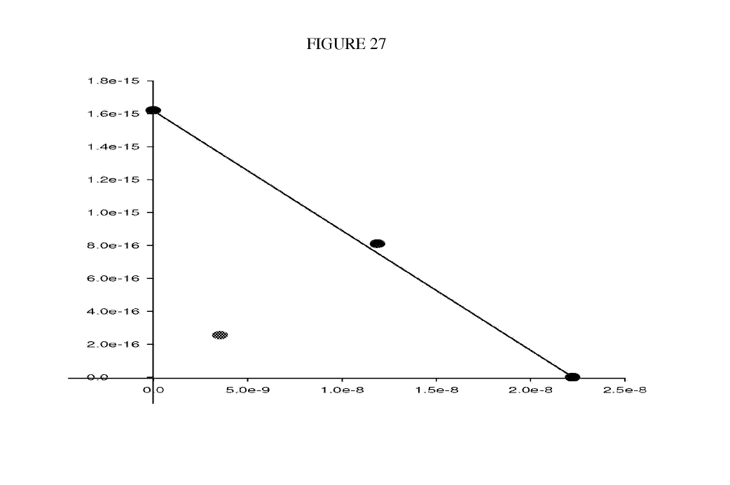

Dose-response effects on mitochondrial swelling to EPI and NICO treatment were pursued. The effective dose (ED) at 30, 40 and 50% of maximal effect were determined by using Michaelis-Menten (M-N) and probabilistic (Probits) analysis. We determined the effects of ED₃₀ EPI and NICO separately and the theoretical effects of the mixture of each compound (equaling a 30 percent of effect) and performed an isobolographic analysis with the data. These results are presented in FIGS. 25-27. As demonstrated, EPI and NICO can limit calcium-induced mitochondrial damage. Co-treatment leads to strong synergistic effects as determined by isobolographic analysis.

### Example 11

To further elucidate the combined effect of epicatechin and nicorandil on physiological function, the rat myocardial I/R model was again used. The purpose was to compare the effects that low doses of the compounds when given either alone or in combination have on infarct size when given repeatedly (2 or 3 times) over the course of 24 h after I/R.

The general methods for the implementation of the rat myocardial IR model are described herein. The total time of myocardial ischemia was 45 min Treatment was administered a total of 1, 2 or 3 times. The initial dose was given15 min prior to reperfusion and then at 12 (in the case of 2× and 3× dosing) and again at 24 h (in the case of 3× dosing) after reperfusion. Epi (0.5 mg/kg) and/or Nico (33 f× g/kg) were mixed in water and given IV using the jugular vein. Control animals only received water injections. Infarct size was examined 48 h after IR using established procedures.

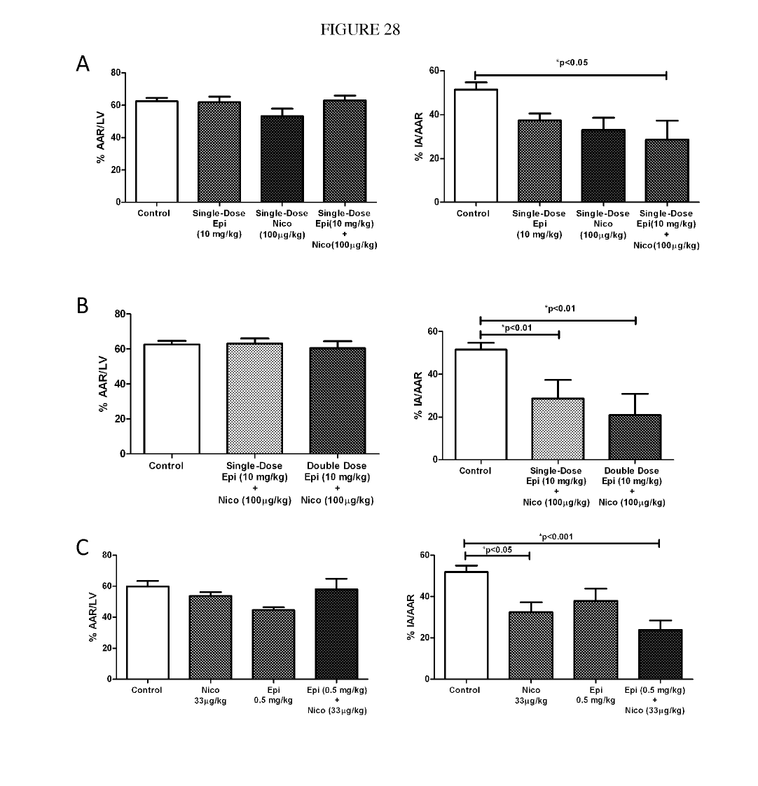

The results are depicted in FIG. 28. Panel A depicts the results obtained with a single dosage of Epi and/or Nico; panel B the results obtained with 2× dosage of the combination; and panel; C the results obtained with a 3× dosage of Epi and/or Nico. Results indicate that Nico alone can reduce infarct size in a significant manner by 37%. Epi alone reduces infarct size by only 27% in a non-significant manner. The combination of both drugs yields a highly significant 54% reduction vs. controls. Thus, repeated low dose Nico+Epi represents a potential useful treatment algorithm where side effects and toxicity are minimized

While the invention has been described and exemplified in sufficient detail for those skilled in this art to make and use it, various alternatives, modifications, and improvements should be apparent without departing from the spirit and scope of the invention. The examples provided herein are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are set forth within the following claims.

1.-39. (canceled)

40. A method of stimulating mitochondrial function in cells, comprising:
administering one or more compounds selected from the group consisting of epicatechin, an epicatechin derivative, catechin, a catechin derivative, nicorandil, and a nicorandil derivative in an amount effective to stimulate mitochondrial function in said cells.

41. A method according to claim 40, wherein said stimulation of mitochondrial function in said cells comprises stimulation of mitochondrial respiration in said cells.

42. A method according to claim 40, wherein said stimulation of mitochondrial function in said cells comprises stimulation of mitochondrial biogenesis in said cells.

43. A method according to claim 40, wherein said administration comprises administering at least 0.1 μM catechin, a catechin derivative, epicatechin or an epicatechin derivative to said cells.

44. A method according to claim 43, wherein said at least 0.1 μM catechin, a catechin derivative, epicatechin or an epicatechin derivative is maintained at least 30 minutes, 1 hour, 3 hours, 12 hours, 24 hours, or 48 hours.

45. A method according to claim 40, wherein said administration comprises administering at least 1 μM catechin, a catechin derivative, epicatechin or an epicatechin derivative to said cells.

46. A method according to claim 45, wherein said at least 1 μM catechin, a catechin derivative, epicatechin or an epicatechin derivative is maintained for at least 30 minutes, 1 hour, 3 hours, 12 hours, 24 hours, or 48 hours.

47. A method according to claim 40, wherein said epicatechin derivative has the structure: 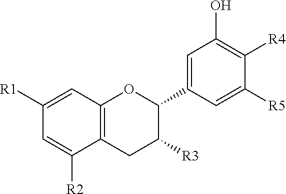
wherein
R1, R2, and R4 are each independently selected from the group consisting of —OH, —O—C1-6 straight or branched chain alkyl, —O—C1-12 arylalkyl, —C1-6 straight or branched chain alkyl, and —C1-12 arylalkyl, wherein each said straight or branched chain alkyl or arylalkyl comprises from 0-4 chain heteroatoms and optionally one or more substituents independently selected from the group consisting of halogen, trihalomethyl, —O—C1-6 alkyl, —NO2, —NH2, —OH, —CH2OH, —CONH2, and —C(O)(OR6) where R6 is H or C1-3 alkyl, provided that at least one of R1, R2, and R4 is not —OH; 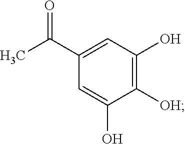
R3 is —OH or and
R5 is H or OH,
or a pharmaceutically acceptable salt thereof.

48. A method according to claim 47, wherein said epicatechin derivative has a structure selected from the groups consisting of

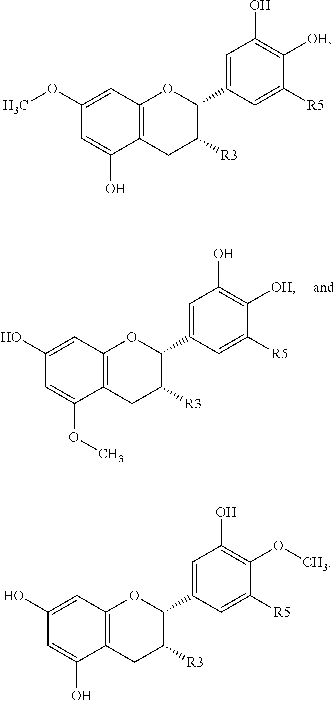

49. A method according to claim 40, wherein said administering step comprises delivering one or more compounds selected from the group consisting of epicatechin, an epicatechin derivative, catechin, a catechin derivative, nicorandil, and a nicorandil derivative to an animal by a parenteral or enteral route in an amount effective to stimulate mitochondrial function in cells of said animal.

50. A method according to claim 49, wherein said animal is a human.

51. A method according to claim 49, wherein said animal is selected for said administering step based on a diagnosis that said animal is suffering from or at immediate risk of suffering from one or more conditions selected from the group consisting of an inborn error of mitochondrial biogenesis or bioenergetics, a dietary deficiency, a vitamin deficiency, diabetes, metabolic syndrome, Friedreich's ataxia, pulmonary hypertension, chronic kidney disease, acute kidney injury, hypertension, dementia, heart failure, obesity, insulin resistance, a muscular condition involving decreased mitochondrial function, impaired cognition related to aging, vascular disease, metabolic impairment or neurodegeneration, and a neurological condition involving decreased mitochondrial function.

52. A method according to claim 49, wherein said animal is selected for said administering step based on age of said animal.

53. A method according to claim 49, wherein said animal is selected for said administering step based on an activity state of said animal.

54. A method according to claim 49, wherein said administering step comprises delivering catechin, a catechin derivative, epicatechin or an epicatechin derivative by an oral route in an amount effective to maintain a plasma concentration of at least 0.1 μM of said compound in said animal for at least 30 minutes, 1 hour, 3 hours, 12 hours, 24 hours, or 48 hours.

55. A method according to claim 49, comprises delivering catechin, a catechin derivative, epicatechin or an epicatechin derivative by an oral route in an amount effective to maintain a plasma concentration of at least 1 μM of said compound in said animal for at least 30 minutes, 1 hour, 3 hours, 12 hours, 24 hours, or 48 hours.

56. A method of treating a condition involving decreased mitochondrial function in an animal, said method comprising:
delivering to said animal one or more compounds selected from the group consisting of epicatechin, an epicatechin derivative, catechin, a catechin derivative, nicorandil, and a nicorandil derivative to an animal by a parenteral or enteral route in an amount effective to stimulate mitochondrial function in cells of said animal.

57. A method according to claim 56, wherein said condition involving decreased mitochondrial function is selected from the group consisting of an inborn error of mitochondrial biogenesis or bioenergetics, a dietary deficiency, a vitamin deficiency, diabetes, metabolic syndrome, Friedreich's ataxia, pulmonary hypertension, chronic kidney disease, acute kidney injury, hypertension, dementia, heart failure, obesity, insulin resistance, a muscular condition involving decreased mitochondrial function, impaired cognition related to aging, vascular disease, metabolic impairment or neurodegeneration, and a neurological condition involving decreased mitochondrial function.

58. A method according to claim 56, wherein said condition involving decreased mitochondrial function is related to the age and/or activity state of said animal.

59. A method according to claim 56, wherein said condition involving decreased mitochondrial function is related to a nutritional state of said animal.

60. A method according to claim 56, wherein said administering step comprises delivering to said animal catechin, a catechin derivative, epicatechin or an epicatechin derivative by an oral route in an amount effective to maintain a plasma concentration of at least 0.1 μM of said compound in said animal for at least 30 minutes, 1 hour, 3 hours, 12 hours, 24 hours, or 48 hours.

61. A method according to claim 56, comprises delivering to said animal catechin, a catechin derivative, epicatechin or an epicatechin derivative by an oral route in an amount effective to maintain a plasma concentration of at least 1 μM of said compound in said animal for at least 30 minutes, 1 hour, 3 hours, 12 hours, 24 hours, or 48 hours.

62. A method for improving muscle structure or function in an animal, comprising:
administering one or more compounds selected from the group consisting of epicatechin, an epicatechin derivative, catechin, a catechin derivative, nicorandil, and a nicorandil derivative to said animal in an amount effective to stimulate mitochondrial function in cells, thereby improving muscle structure or function in said animal.

63. A method for improving mitochondrial effects associated with exercise in an animal, comprising:
administering one or more compounds selected from the group consisting of epicatechin, an epicatechin derivative, catechin, a catechin derivative, nicorandil, and a nicorandil derivative to said animal in an amount effective to stimulate mitochondrial function in cells, thereby improving mitochondrial effects associated with exercise in said animal.

64. A method for enhancing the capacity for exercise in an animal, comprising: administering one or more compounds selected from the group consisting of epicatechin, an epicatechin derivative, catechin, a catechin derivative, nicorandil, and a nicorandil derivative to said animal in an amount effective to stimulate mitochondrial function in cells, thereby enhancing the capacity for exercise in said animal.

65. A method for enhancing muscle health and function in response to exercise in an animal, comprising:
administering one or more compounds selected from the group consisting of epicatechin, an epicatechin derivative, catechin, a catechin derivative, nicorandil, and a nicorandil derivative to said animal in an amount effective to stimulate mitochondrial function in cells, thereby enhancing muscle health and function in response to exercise in said animal.

66. A method for enhancing muscle health and function in a clinical setting of restricted capacity for exercise in an animal, comprising:
administering one or more compounds selected from the group consisting of epicatechin, an epicatechin derivative, catechin, a catechin derivative, nicorandil, and a nicorandil derivative to said animal in an amount effective to stimulate mitochondrial function in cells, thereby enhancing muscle health and function in said animal.

67. A method for enhancing recovery of muscles from vigorous activity or from injury associated with vigorous or sustained activity in an animal, comprising:
administering one or more compounds selected from the group consisting of epicatechin, an epicatechin derivative, catechin, a catechin derivative, nicorandil, and a nicorandil derivative to said animal in an amount effective to stimulate mitochondrial function in cells, thereby enhancing recovery of muscles in said animal.

68. A method according to one of claim 56, 62, 63, 64, 65, 66, or 67 wherein said administration comprises administering at least 0.1 μM catechin, a catechin derivative, epicatechin or an epicatechin derivative to said cells.

69. A method according to claim 56, 62, 63, 64, 65, 66, or 67, wherein said method comprises administering epicatechin or an epicatechin derivative which is at least 90% pure relative to other compounds selected from the group consisting of epicatechin, an epicatechin derivative, catechin, or a catechin derivative.

70-87. (canceled)
